(19) United States
(12) Patent Application Publication
(10) Pub. No.: US 2013/0029330 A1
(43) Pub. Date: Jan. 31, 2013

(54) MUTANT LDL RECEPTOR GENE

(71) Applicants: Said M. Shawar, Manama (BH); Ahmad R. Ramadan, Manama (BH); Mohammad A. Al-Drees, Manama (BH); Hasan Ali Najat, Manama (BH)

(72) Inventors: Said M. Shawar, Manama (BH); Ahmad R. Ramadan, Manama (BH); Mohammad A. Al-Drees, Manama (BH); Hasan Ali Najat, Manama (BH)

(73) Assignee: ARABIAN GULF UNIVERSITY, Manama (BH)

(21) Appl. No.: 13/518,548

(22) PCT Filed: Dec. 22, 2010
(86) PCT No.: PCT/EP2010/070566

§ 371 Date: Oct. 10, 2012

(30) Foreign Application Priority Data
Dec. 22, 2009 (GB) 0922377.7

(51) Int. Cl. C12N 15/12; G01N 21/64; C12N 15/63; C07H 21/00; C12N 1/21; C12N 5/10; C07K 14/705; C07K 16/28; C12Q 1/68; C12N 15/79

(52) U.S. Cl. 435/6.11; 435/7.24; 536/23.5; 435/320.1; 435/252.3; 435/325; 530/350; 530/387.9; 536/24.31

(57) ABSTRACT

There is disclosed a method of identifying individuals susceptible to familial hypercholesterolemia and which method comprises identifying in a sample from said individual at least one polymorphism at position 1706-2 of the coding region (41902 of the genomic DNA) in the low density lipoprotein receptor gene, and wherein the presence of at least one said polymorphism is indicative of said individual being of a higher susceptibility to familial hypercholesterolemia.

The present invention is concerned with the low density lipoprotein receptor (LDLR) gene. More particularly, the invention concerns nucleic acid molecules that comprise a novel mutation in the LDLR gene and methods to screen for the presence or absence of mutations or polymorphisms in the LDLR gene.

### BACKGROUND OF THE INVENTION

Familial hypercholesterolemia (FHC) is a monogenic autosomal dominant disorder caused by defects in the gene coding for LDLR. The worldwide prevalence of heterozygous FHC is 1 in 500 and 1 per million for the homozygous. However, prevalence of heterozygous FHC could be as high as 1 in 50 in communities with a ‘founder gene’, such as the French Canadians and Lebanese Christians (1, 2, 3).

Many genetic disorders with founder mutation(s) are known. For example high mutation frequencies of the Tay-Sachs (4) and mutations in BRAC-1 and 2 (5) are known in Ashkenazi Jews. The Lebanese allele of low density lipoprotein receptor (LDLR) that causes familial hypercholesterolemia is prevalent among Lebanese Christians and DF508 allele in cystic fibrosis is prevalent across Europe (6). Founder mutations can remain restricted to a very small geographical area or a small population (7, 8). However, this generalization is incorrect simply because sickle cell anemia is internationally affecting individuals mainly with African descent (9).

FHC is a major risk factor for premature coronary heart disease (CHD). Indeed, FHC gives a 100-fold excess risk of CHD in young men resulting in more than 200,000 worldwide deaths annually (10, 11, 12). The number of LDLR mutations exceeds 1600 (13). Limited studies are reported on the nature of mutations among Arabs in general or Arabs in the Gulf region (14).

The present inventors have identified a single nucleotide substitution in the low density lipoprotein receptor (LDLR) gene. The substitution in the acceptor splice site of LDLR intron 11 leads to several molecular events among which is the use of a new cryptic splice site, transcript deletion, a frame shift, premature stop codon, low mRNA expression, protein truncation and low protein surface expression. The mutation leads to low gene expression of LDLR mRNA due to non-sense mediated decay (NMD) and low protein surface receptor through several consecutive cellular and molecular events. It is believed that this mutation represents a founder effect and may be used in mass screenings and rapid and early diagnosis of all descendants sharing this allele.

This substitution was found in two unrelated Arab Gulf families (tribes) who descended from Ismail (Ishmael), the father of Arabs according to genealogy, history and tradition (15). This mutation is believed to be a founder mutation and could be used for rapid population screening, prenatal diagnosis and pre-implantation genetic diagnosis (16). The mutation has been designated “The LDLR Arabic Allele”.

Therefore, there is provided by the present invention a method of identifying individuals susceptible to familial hypercholesterolemia (FHC) and which method comprises identifying in a DNA sample from said individual at least one allelic polymorphism at position 1706-2 of the coding region (41902 of the genomic DNA) in the low density lipoprotein receptor gene, and wherein the presence of at least one said polymorphism is indicative of said individual being of a higher susceptibility to FHC. In a preferred embodiment, the polymorphism is a substitution and more preferably a substitution of the Adenosine (A) nucleotide for a Thymine (T). The polymorphism is indicated as being present at position 1706-2 of the coding region. Accordingly, the polymorphism occurs in intron 11 two bases before the beginning of the wild type coding region which occurs at position 1706. This may also be referred to as position 41902 of the genomic DNA according to the NCIB database.

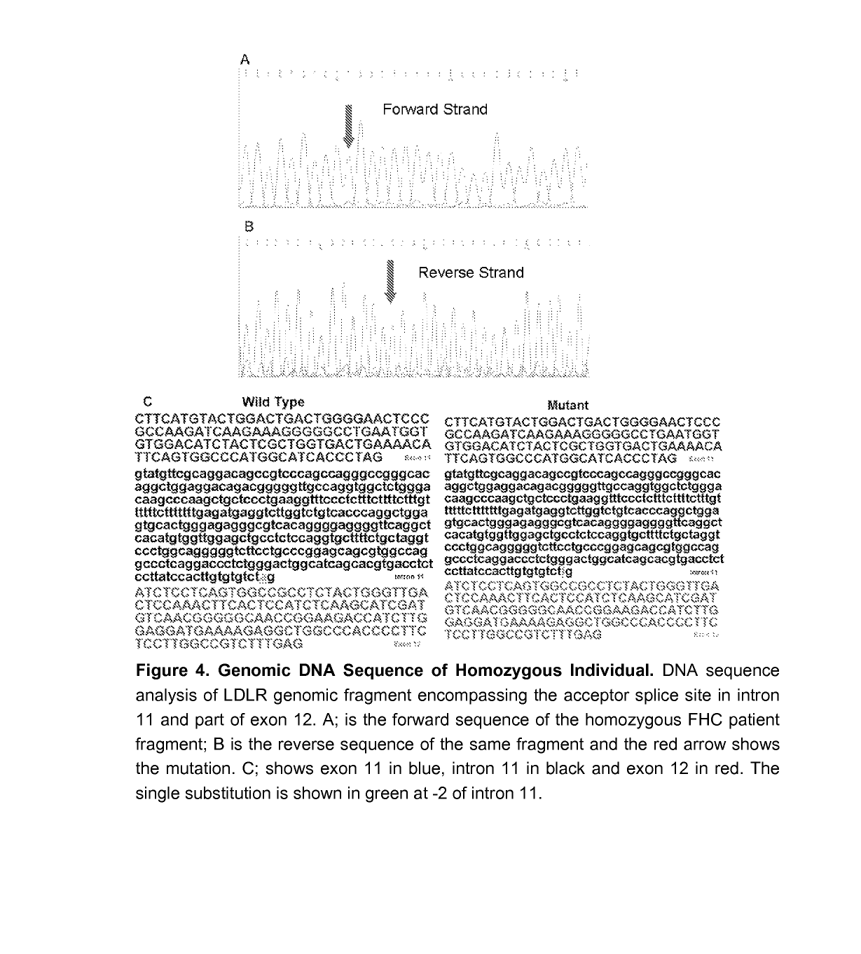

The polymorphism of the present invention therefore represents a polymorphic substitution at a singe nucleotide position 1706-2 (41902 of the genomic DNA) which is located in intron 11 in the wild-type genomic DNA. Since introns do not occur n the eventual coding sequence the polymorphic site is indicated as being present in the penultimate nucleotide preceding the coding region which coding region begins at position 1706, as indicated in FIG. 4. Therefore, the mutation occurs in the penultimate nucleotide in the splice acceptor site of intron 11 which is 2 nucleotides before the coding region or according to other terminology, (as set out in Graham et al., Atherosclerosis, 18:331-340 (2007), intervening sequence 11 (ivs11). The full wild type sequence of the human LDLR gene can be found at http://www.umd.necker.fr/LDLR/genomic.html#ancre538417. A partial sequence of the wild type genomic DNA identifying the A at position 1706-2 (41902 genomic DNA) is as shown in SEQ ID No: 1. The mutant according to the invention has been designated the Arabic allele and is shown herein again at position 1706-2 (41902 of the genomic DNA) in SEQ ID No. 2. The resulting cDNA of the transcribed genomic fragment and the translated polypeptide sequence are provided as SEQ ID NO: 3 and 4 respectively. The polymorphism may be identified using many known techniques in the art as described in greater detail herein and which may include any of polymerase chain reaction hybridization, Southern blotting onto membrane, digestion with nucleases, restriction fragment length polymorphism, or direct sequencing, or combinations thereof. In one embodiment the polymorphism is detected by PCR using forward and reverse primers of exon 11 and 12 respectively of the LDLR gene. In one embodiment the primers used are 5′-CAG CTA TTC TCT GTC CTC CCA CCA G (SEQ ID NO: 5) and 5′-CGTACGAGATGCAAGCACTTAGGTG (SEQ ID NO: 6); or 5′-CCAGGTGCTTTTCTGCTAGG (SEQ ID NO: 7) and 5′-TCACTCCATCTCAAGCATCG (SEQ ID NO: 8) or 5′-CCTCTCCAGGTGCTTTTCTG (SEQ ID NO: 9) and 5′-TCACTCCATCTCAAGCATCG (SEQ ID NO: 8).

Another aspect of the invention provides a nucleic acid, such as a probe or primer, which hybridizes under high or low stringency conditions to a nucleic acid having all or a portion of a nucleic acid sequences according to the invention. Alternatively, high resolution melt (HRM) in Real Time PCR can be employed to identify the mutation. This technique depends on changing the stringency conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

Isolated nucleic acids encoding the polypeptide of the invention, and having a sequence which differs from a nucleotide sequence shown in SEQ ID NO: 2 or 3 due to degeneracy in the genetic code are also within the scope of the invention. Such nucleic acids encode structurally equivalent proteins but differ in sequence from the sequence of SEQ ID NO: 2 or 3 due to degeneracy in the genetic code. Degeneracy means that a number of amino acids are designated by more than one triplet.

The nucleic acid molecule of the present invention may comprise the full length nucleotide sequence (SEQ ID No: 2) incorporating the polymorphism or that of the open-reading frame identified in SEQ ID No: 3 or sequences complementary thereto, or sequences exhibiting at least 70%, 75%, 80%, 85%, 90%, 95% or 99% sequence identity or homology to the sequences of SEQ ID No. 2 or 3. In another embodiment, the present invention provides nucleic acid molecules that code for a polypeptide having an amino acid sequence exhibiting any of at least 70%, 75%, 80%, 85%, 90%, 95% or 99% homology or identity to the amino acid sequence according to SEQ ID No: 4.

The term “isolated” refers to a nucleic acid substantially free of cellular material or culture medium when produced by recombinant DNA techniques, or chemical precursors or other chemicals when chemically synthesized. An “isolated” nucleic acid molecule is also free of sequences which naturally flank the nucleic acid (i.e., sequences located at the 5′ and 3′ ends of the nucleic acid) in the organism from which the nucleic acid is derived. The term “nucleic acid” molecule is intended to include DNA and RNA and can be either double stranded or single stranded. In one embodiment, the nucleic acid is a cDNA comprising a nucleotide sequence shown in SEQ ID NO: 3. In another embodiment, the nucleic acid is a genomic DNA comprising the nucleotide sequence shown in SEQ ID NO: 2. In another embodiment, the nucleic acid encodes a protein comprising an amino acid sequence shown in SEQ ID NO: 4.

The invention includes nucleic acids having substantial sequence homology with the nucleotide sequence shown in SEQ ID NO: 2 or SEQ ID NO 3 or encoding proteins having substantial homology to the amino acid sequence shown in SEQ ID NO: 4. Homology refers to sequence similarity between sequences and can be determined by comparing a position in each sequence which may be aligned for purposes of comparison. When a position in the compared sequence is occupied by the same nucleotide base or amino acid, then the molecules are homologous at that position. A degree of homology between sequences is a function of the number of matching or homologous positions shared by the sequences.

The term “sequences having substantial sequence homology” means those nucleotide and amino acid sequences which have slight or inconsequential sequence variations from the sequences disclosed in SEQ ID No:2 and SEQ ID No: 3, i.e. the homologous nucleic acids function in substantially the same manner to produce substantially the same polypeptides as the actual sequences and either incorporate the polymorphism at position 1706-2 or 41902 or arise as a result of the different splice acceptor site, for example as set out in SEQ ID No. 3. Alternative splice variants corresponding to a cDNA of the invention are also encompassed.

“Percent (%) amino acid sequence identity” with respect to the polypeptide sequences identified herein is defined as the percentage of amino acid residues in a candidate sequence that are identical with the reference amino acid residues in the polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Percent amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the polypeptide of interest having a sequence derived from the mutant LDL polypeptide and the comparison amino acid sequence of interest as determined by WU-BLAST-2 by (b) the total number of amino acid residues of the polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the polypeptide of interest.

Percent amino acid sequence identity may also be determined using the sequence comparison program NCBI-BLAST2 (Altschul et al., Nucleic Acids Res. 25:3389-3402 (1997)). NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62

The present invention also relates to an antisense nucleic acid, or oligonucleotide fragment thereof, of a nucleic acid of the invention incorporating the Arabic allele. An antisense nucleic acid can comprise a nucleotide sequence which is complementary to a coding strand of a nucleic acid, e.g. complementary to an mRNA sequence or a coding region of a genomic DNA, constructed according to the rules of Watson and Crick base pairing, and can hydrogen bond to the coding strand of the nucleic acid.

The present invention also provides recombinant vectors comprising nucleic acid molecules of the invention as described herein. These recombinant vectors may be plasmids. In other embodiments, these recombinant vectors are prokaryotic or eukaryotic expression vectors. The nucleic acid molecules of the invention may also be operatively linked to a regulatory control sequence.

The nucleic acids of the present invention can be incorporated into a recombinant expression vector using techniques known in the art, thus ensuring good expression of the encoded protein or part thereof. The recombinant expression vectors are “suitable for transformation of a host cell”, which means that the recombinant expression vectors contain a nucleic acid or an oligonucleotide fragment thereof of the invention in addition to a regulatory sequence, selected on the basis of the host cells to be used for expression, which is operatively linked to the nucleic acid or oligonucleotide fragment. Operatively linked is intended to mean that the nucleic acid is linked to a regulatory sequence in a manner which allows expression of the nucleic acid. Therefore, nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Regulatory sequences are art-recognized and are selected to direct expression of the desired protein in an appropriate host cell. Accordingly, the term regulatory sequence includes promoters, enhancers and other expression control elements. Such regulatory sequences are known to those skilled in the art.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Expression of these recombinant expression vectors is carried out in prokaryotic or eukaryotic cells using standard molecular biology techniques.

The recombinant expression vectors of the invention can be used to make a transformant host cell including the recombinant expression vector. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation, microinjection or any other known technique.

The present invention further provides host cells comprising a nucleic acid of the invention.

Nucleic acids of the invention can be used to generate either transgenic animals or “knock in-knock out” animals that, in turn, may be useful in further understanding the mechanism of action of LDLR. A transgenic mammal (e.g. a rat or a mouse) is a mammal having cells that contain a transgene, which was introduced into the mammal or an ancestor of the mammal at a prenatal, e.g. an embryonic stage. A transgene is a DNA molecule which is integrated into the genome of a cell from which a transgenic animal develops. The nucleic acid molecules can be contained within recombinant vectors such as plasmids, phages, viruses, transposons, cosmids or artificial chromosomes. Such vectors can also include regulatory elements that control the replication and expression of the LDLR nucleic acid sequences. The vectors can also contain sequences that allow for the screening or selection of cells containing the vector. Such screening or selection sequences can include antibiotic resistance genes. The recombinant vectors can be prokaryotic expression vectors or eukaryotic expression vectors. The nucleic acid can be linked to a heterologous promoter.

A nucleic acid molecule is a “polynucleotide” which is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”. A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The present invention also relates to a method for preparing isolated polypeptides encoded by the LDLR Arabic allele which method comprises culturing a transformed host cell including a recombinant expression vector in a suitable medium until said polypeptide is formed, and subsequently isolating the polypeptide. The steps in such a method represent standard laboratory techniques.

Host cells comprising a nucleic acid of the invention are also provided. The host cells can be prepared by transfecting a nucleic acid of the invention into a cell using transfection techniques known in the art. These techniques include calcium phosphate co-precipitation, microinjection, electroporation and liposome-mediated gene transfer.

The present invention further provides an antibody or antigen-binding fragment specific for an epitope of a polypeptide encode by the LDLR Arabic allele of the invention. The antibodies or antigen-binding fragments may be polyclonal or monoclonal. Such polyclonal or monoclonal antibodies or antigen-binding fragments may be coupled to a detectable substance. The antibodies can be incorporated in compositions suitable for administration in a pharmaceutically acceptable carrier. Such antibodies may also be used to identify the polypeptide encoded by the LDLR Arabic allele and so provides another mechanism for identifying or screening individuals expressing the LDLR Arabic allele.

Immunogenic portions of the polypeptides encoded by the Arabic allele can be used to prepare specific antibodies which discriminate between those polypeptide encoded by the Arabic allele and wild type LDLR. Antibodies can be prepared which bind to an epitope in a region of the polypeptide. The term antibody is also intended to include fragments which are specifically reactive with the polypeptide. Antibodies can be fragmented using conventional techniques, for example, F(ab′)2 fragments can be generated by treating antibody with pepsin. The resulting F(ab′)2 fragment can be treated to reduce disulfide bridges to produce Fab′ fragments. Polyclonal antibodies are antibodies that are derived from different B-cell lines and are a mixture of immunoglobulin molecules secreted against a specific antigen, each recognising a different epitope. Monoclonal antibodies are antibodies that are identical because they were produced by one type of B-cell and are all clones of a single parent cell. Standard techniques are used to produce polyclonal antibodies. Routine procedure based on the hybridoma technique originally developed by Kohler and Milstein (Nature 256: 495-497 (1975)) is used to produce monoclonal antibodies.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng. 8(10):1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein in relation to a polypeptide or antibody refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368: 856-859 (1994); Morrison, Nature 368:812-13 (1994); Fishwild et al., Nature Biotechnology 14:845-51 (1996); Neuberger, Nature Biotechnology 14:826 (1996); Lonberg and Huszar, Intern. Rev. Immunol. 13:65-93 (1995).

The phrase “biological sample”, as used herein, is intended to mean any sample comprising a cell, a tissue, or a bodily fluid obtained from an organism that can be assayed using the methods of the present invention to detect LDLR gene polymorphisms. An example of such a biological sample includes a “body sample” obtained from a human patient. A “body sample” includes, but is not limited to, blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

As used herein, “elevated risk of or increased susceptibility of developing familial hypercholesterolemia (FHC)” refers to an individual with a genotype predictive of a greater likelihood of having or developing FHC as compared to another individual with a different genotype. Specifically, an individual with at least a single allele with a substitution of nucleotide A to T at position 1706-2 (41902 of the genomic DNA sequence), the so called Arabic allele as designated herein, will be at increased risk of having FHC than an individual who does not have the allele. Similarly, an individual that is homozygous for the two Arabic alleles will have severe FHC.

An “allele,” as used herein, refers to one specific form of a genetic sequence (such as a gene) within a cell, an individual or within a population, the specific form differing from other forms of the same gene in the sequence of at least one, and frequently more than one, variant sites within the sequence of the gene. The sequence may or may not be within a gene. The sequences at these variant sites that differ between different alleles are termed “variances”, “polymorphisms”, or “mutations”. At each autosomal specific chromosomal location or “locus”, an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

“Polymorphism,” as used herein, refers to the occurrence of two or more genetically determined alternative sequences or alleles in a population. A polymorphic marker or site is the locus at which divergence occurs. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

The term “genotyping,” as used herein, refers to the determination of the genetic information an individual carries at one or more positions in the genome. For example, genotyping may comprise the determination of which allele or alleles an individual carries for a single polymorphism or the determination of which allele or alleles an individual carries for a plurality of polymorphisms. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the A allele, and those individuals who have one copy of each allele are heterozygous. The array may be designed to distinguish between each of these three possible outcomes. A polymorphic location may have two or more possible alleles and the array may be designed to distinguish between all possible combinations.

A “polynucleotide” means a single strand or parallel and anti-parallel strands of a nucleic acid. Thus, a polynucleotide may be either a single-stranded or a double-stranded nucleic acid. A polynucleotide is not defined by length and thus includes very large nucleic acids, as well as short ones, such as an oligonucleotide.

The term “nucleic acid” typically refers to large polynucleotides. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 50 nucleotides. It will be understood that when a nucleotide sequence is represented by a DNA sequence (i.e., A, T, Q C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”. Sequences on a DNA strand that are located 5′ to a reference point on the DNA are referred to as “upstream sequences”. Sequences on a DNA strand that are 3′ to a reference point on the DNA are referred to as “downstream sequences.”

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. Typical uses of primers include, but are not limited to, sequencing reactions and amplification reactions. A primer is typically single-stranded, but may be double-stranded. Primers are typically deoxyribonucleic acids, but a wide variety of synthetic and naturally-occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., detectable moieties, such as chromogenic, radioactive or fluorescent moieties, or moieties for isolation, e.g., biotin.

“Probe” refers to a polynucleotide that is capable of specifically hybridizing to a designated sequence of another polynucleotide. “Probe” as used herein encompasses oligonucleotide probes. A probe may or may not provide a point of initiation for synthesis of a complementary polynucleotide. A probe specifically hybridizes to a target complementary polynucleotide, but need not reflect the exact complementary sequence of the template. In such a case, specific hybridization of the probe to the target depends on the stringency of the hybridization conditions. For use in SNP detection, some probes are allele-specific, and hybridization conditions are selected such that the probe binds only to a specific SNP allele. Probes can be labeled with, e.g., detectable moieties, such as chromogenic, radioactive or fluorescent moieties, and used as detectable agents.

As used herein in relation to nucleic acids, “label” refers to a group covalently attached to a polynucleotide. The label may be attached anywhere on the polynucleotide but is preferably attached at one or both termini of the polynucleotide. The label is capable of conducting a function such as giving a signal for detection of the molecule by such means as fluorescence, chemiluminescence, and electrochemical luminescence. Alternatively, the label allows for separation or immobilization of the molecule by a specific or non-specific capture method (Andrus, 1995, In: PCR 2: A Practical Approach, McPherson et al. (Eds) Oxford University Press, Oxford, England, pp. 39-54). Labels include, but are not limited to, fluorescent dyes, such as fluorescein and rhodamine derivatives (U.S. Pat. Nos. 5,188,934 and 5,366,860), cyanine dyes, haptens, and energy-transfer dyes (Clegg, 1992, Methods Enzymol. 211:353-388; Cardullor et al., 1988, PNAS 85:8790-8794).

The term “target sequence”, “target nucleic acid” or “target” refers to a nucleic acid of interest. The target sequence may or may not be of biological significance. Typically, though not always, it is the significance of the target sequence that is being studied in a particular experiment. As non-limiting examples, target sequences may include regions of genomic DNA that are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854; 5,445,934; 5,744,305; 5,677,195; 5,800,992; 6,040,193 and 5,424,186, and Fodor et al., 1991, Science 251:767-777, each of which is incorporated by reference in its entirety for all purposes.

Arrays may generally be produced using a variety of techniques, such as mechanical synthesis methods or light directed synthesis methods that incorporate a combination of photolithographic methods and solid-phase synthesis methods. Techniques for the synthesis of these arrays using mechanical synthesis methods are described in, e.g., U.S. Pat. Nos. 5,384,261 and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358; 5,789,162; 5,708,153; 6,040,193 and 5,800,992, which are hereby incorporated by reference in their entirety for all purposes.)

Arrays may be packaged in such a manner as to allow for diagnostic use or can be an all-inclusive device. Preferred arrays are commercially available from Affymetrix (Santa Clara, Calif.) under the brand name GeneChip RTM, and are directed to a variety of purposes, including genotyping and gene expression monitoring for a variety of eukaryotic and prokaryotic species.

“Amplification” refers to any means by which a polynucleotide sequence is copied and thus expanded into a larger number of polynucleotide sequences, e.g., by reverse transcription, polymerase chain reaction or ligase chain reaction, among others.

“Hybridization probes,” as used herein, are oligonucleotides capable of binding in a base-specific manner to a complementary strand of nucleic acid. Such probes include peptide nucleic acids, as described in Nielsen et al., 1991, Science 254:1497-1500, and other nucleic acid analogs and nucleic acid mimetics. See U.S. patent application Ser. No. 08/630,427.

An “individual,” as used herein, is not limited to a human being, but may also include other organisms including but not limited to mammals.

### Nucleic Acids: Primers

The present invention encompasses isolated nucleic acids useful in the practice of the methods of the invention. Specifically, the present invention encompasses primers useful in the amplification of polymorphisms in the LDLR gene. Each primer should be sufficiently long to initiate or prime the synthesis of extension DNA products in the presence of an appropriate polymerase and other reagents. Appropriate primer length is dependent on many factors, as is well known; typically, in the practice of the present invention, a primer will be used that contains 10-30 nucleotide residues. Short primer molecules generally require lower reaction temperatures to form and to maintain the primer-template complexes that support the chain extension reaction.

The primers used need to be substantially complementary to the nucleic acid containing the selected sequences to be amplified, i.e. the primers must bind to, i.e. hybridize with, nucleic acid containing the selected sequence (or its complement). The primer sequence need not be entirely an exact complement of the template; for example, a non-complementary nucleotide fragment or other moiety may be attached to the 5′ end of a primer, with the remainder of the primer sequence being complementary to the selected nucleic acid sequence. Primers that are fully complementary to the selected nucleic acid sequence are preferred and typically used.

Generally, primers will be between about 10 and 30 nucleotides in length. They are preferably chosen to hybridize to a unique DNA sequence in the genome so as to maximize the desired location hybridization that will occur. In one embodiment the primer pairs used are 5′-CAG CTA TTC TCT GTC CTC CCA CCA G (SEQ ID NO: 5) and 5′-CGTACGAGATGCAAGCACTTAGGTG (SEQ ID NO: 6); or 5′-CCAGGTGCTTTTCTGCTAGG (SEQ ID NO: 7) and 5′-TCACTCCATCTCAAGCATCG (SEQ ID NO: 8) or 5′-CCTCTCCAGGTGCTTTTCTG (SEQ ID NO: 9) and 5′-TCACTCCATCTCAAGCATCG (SEQ ID NO: 8).

The target sequence or target nucleic acid may be a portion of a gene, a regulatory sequence, genomic DNA, cDNA, and RNA (including mRNA and rRNA). Genomic DNA samples are usually amplified before being brought into contact with a probe. Genomic DNA can be obtained from any biological sample, including, by way of non-limiting example, tissue source or circulating cells (other than pure red blood cells). For example, convenient sources of genomic DNA include whole blood, semen, saliva, tears, urine, fecal material, sweat, buccal cells, skin and hair. Amplification of genomic DNA containing a polymorphic site generates a single species of target nucleic acid if the individual from which the sample was obtained is homozygous at the polymorphic site, or two species of target molecules if the individual is heterozygous. RNA samples also are often subject to amplification. In this case, amplification is typically preceded by reverse transcription. Amplification of all expressed mRNA can be performed as described in, for example, PCT Publication Nos. WO96/14839 and WO97/01603, which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.

An isolated nucleic acid of the present invention can be produced using conventional nucleic acid synthesis or by recombinant nucleic acid methods known in the art (2001, Molecular Cloning: A Laboratory Manual, Cold Spring Harbor Laboratory Press, New York) and Ausubel et al. (2001, Current Protocols in Molecular Biology, Green & Wiley, New York).

### Tags

In one embodiment of the invention, an isolated nucleic acid of the invention comprises a covalently linked tag. By way of a non-limiting example, an isolated nucleic acid of the present invention may comprise a primer, an oligonucleotide, and a target sequence. That is, the invention encompasses a chimeric nucleic acid wherein the isolated nucleic acid sequence comprises a tag molecule. Such tag molecules are well known in the art and include, for instance, a ULS reagent that reacts with the N-7 position of guanine residues, an amine-modified nucleotide, a 5-(3-aminoallyl)-dUTP, an amine-reactive succinimidyl ester moiety, a biotin molecule, ³³P, ³²P, fluorescent labels such as fluorescein (FITC), 5,6-carboxymethyl fluorescein, Texas Red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7.

However, the invention should in no way be construed to be limited to the nucleic acids encoding the above-listed tags. Rather, any tag that may function in a manner substantially similar to these tag polypeptides should be construed to be included in the present invention.

The isolated nucleic acid comprising a tag can be used to localize an isolated nucleic acid, for example, within a cell, a tissue, and/or a whole organism (e.g., a mammalian embryo), detect an isolated nucleic acid, for example, in a cell, and to study the role(s) of an isolated nucleic acid in a cell. Further, addition of a tag facilitates isolation and purification of the isolated nucleic acid.

### Methods of Identifying LDLR Polymorphisms

A number of methods are available for analysis of polymorphisms. Assays for detection of polymorphisms or mutations fall into several categories, including but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays may be useful in the present invention, and are described in relationship to detection of the LDLR gene polymorphism according to the invention.

### 1. Direct Sequencing Assays

In some embodiments of the present invention, polymorphisms are detected using a direct sequencing technique. In these assays, DNA samples are first isolated from a subject using any suitable method. In some embodiments, the region of interest is cloned into a suitable vector and amplified by growth in a host cell (e.g., a bacterium). In other embodiments, DNA in the region of interest is amplified using PCR.

Following amplification, DNA in the region of interest (e.g., the region containing the polymorphism of interest) is sequenced using any suitable method, including but not limited to manual sequencing using radioactive marker nucleotides, or automated sequencing. The results of the sequencing are displayed using any suitable method. The sequence is examined and the presence or absence of a given polymorphism is determined.

### 2. PCR Assays

In some embodiments of the present invention, polymorphisms are detected using a PCR-based assay. In some embodiments, the PCR assay comprises the use of oligonucleotide primers to amplify a fragment containing the polymorphism of interest.

Amplification of a target polynucleotide sequence may be carried out by any method known to the skilled artisan. See, for instance, Kwoh et al. (1990, Am. Biotechnol. Lab. 8:14-25) and Hagen-Mann, et al., (1995, Exp. Clin. Endocrinol. Diabetes 103: 150-155). Amplification methods include, but are not limited to, polymerase chain reaction (“PCR”) including RT-PCR, strand displacement amplification (Walker et al., 1992, PNAS, 89:392-396; Walker et al., 1992, Nucleic Acids Res. 20: 1691-1696), strand displacement amplification using Phi29 DNA polymerase (U.S. Pat. No. 5,001,050), transcription-based amplification (Kwoh et al., 1989, PNAS 86:1173-1177), self-sustained sequence replication (“3SR”) (Guatelli et al., 1990, PNAS 87:1874-1878; Mueller et al., 1997, Histochem. Cell Biol. 108:431-437), the Q.beta. replicase system (Lizardi et al., 1988, BioTechnology 6:1 197-1202; Cahill et al., 1991, Clin. Chem. 37:1482-1485), nucleic acid sequence-based amplification (“NASBA”) (Lewis, 1992, Gen. Eng. News 12 (9): 1), the repair chain reaction (“RCR”) (Lewis, 1992, supra), and boomerang DNA amplification (or “BDA”) (Lewis, 1992, supra). PCR is the preferred method of amplifying the target polynucleotide sequence.

PCR may be carried out in accordance with known techniques. See, e.g., Bartlett et al., eds., 2003, PCR Protocols Second Edition, Humana Press, Totowa, N.J. and U.S. Pat. Nos. 4,683,195; 4,683,202; 4,800,159 and 4,965,188. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained.

The amplified target polynucleotide may be used in one of the detection assays described elsewhere herein to identify the LDLR gene polymorphism (Arabic allele) present in the amplified target polynucleotide sequence.

### 3. Fragment Length Polymorphism Assays

In some embodiments of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

In one embodiment of the present invention, fragment sizing analysis is carried out using the Beckman Coulter CEQ 8000 genetic analysis system, a method well-known in the art for microsatellite polymorphism determination.

a. RFLP Assay

In some embodiments of the present invention, polymorphisms may be detected using a restriction fragment length polymorphism assay (RPLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from control subjects not expressing the Arabic allele.

b. CFLP Assay

In other embodiments, polymorphisms are detected using a CLEAVASE fragment length polymorphism assay (CFLP; Third Wave Technologies, Madison, Wis.; see e.g., U.S. Pat. No. 5,888,780). This assay is based on the observation that, when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given polymorphism. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### 4. Hybridization Assays

In other embodiments of the present invention, polymorphisms may be detected by hybridization assay. In a hybridization assay, the presence or absence of a given polymorphism or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.

In a preferred embodiment, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, polymerase chain reaction (PCR) with labeled primers or labeled nucleotides will provide a labeled amplification product. In another embodiment, transcription amplification using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase (TdT).

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies; magnetic beads (e.g., Dynabeads™); fluorescent dyes (e.g., fluorescein, Texas Red, rhodamine, green fluorescent protein, and the like); radiolabels (e.g., ³H, ¹²⁵I, ³⁵S, ¹⁴C, or ³²P); phosphorescent labels; enzymes (e.g., horse radish peroxidase, alkaline phosphatase and others commonly used in an ELISA); and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149 and 4,366,241, each of which is hereby incorporated by reference in its entirety for all purposes.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target nucleic acid(s) prior to, or after the hybridization. So-called “direct labels” are detectable labels that are directly attached to or incorporated into the target nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids. See Tijssen, 1993, Laboratory Techniques in Biochemistry and Molecular Biology, Vol. 24: Hybridization with Nucleic Acid Probes, which is hereby incorporated by reference in its entirety for all purposes.

a. Direct Detection of Hybridization

In some embodiments, hybridization of a probe to the sequence of interest (e.g., polymorphism) is detected directly by visualizing a bound probe (e.g., a Northern or Southern assay; See e.g., Ausabel et al. (Eds.), 1991, Current Protocols in Molecular Biology, John Wiley & Sons, NY. In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

b. Detection of Hybridization Using “DNA Chip” Assays

In some embodiments of the present invention, polymorphisms and/or differences in levels of gene expression (e.g., mRNA) are detected using a DNA chip hybridization assay. In this assay, a series of oligonucleotide probes are affixed to a solid support. The oligonucleotide probes are designed to be unique to a given polymorphism. The DNA sample of interest is contacted with the DNA “chip” and hybridization is detected.

In some embodiments, the DNA chip assay is a GeneChip (Affymetrix, Santa Clara, Calif., see e.g., U.S. Pat. No. 6,045,996) assay. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (see e.g., U.S. Pat. No. 6,068,818). Through the use of microelectronics, Nanogen's technology enables the active movement and concentration of charged molecules to and from designated test sites on its semiconductor microchip. DNA capture probes unique to a given polymorphism or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (see e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on an X/Y translation stage under a set of four piezoelectric nozzles, one for each of the four standard DNA bases. The translation stage moves along each of the rows of the array, and the appropriate reagent is delivered to each of the reaction site. For example, the A amidite is delivered only to the sites where amidite A is to be coupled during that synthesis step and so on. Common reagents and washes are delivered by flooding the entire surface followed by removal by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif., see e.g., PCT Publications WO99/67641 and WO00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given polymorphism or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, genomic profiles are generated using an assay that detects hybridization by enzymatic cleavage of specific structures (INVADER assay, Third Wave Technologies; see e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the dequenched fluorescein labeled product may be detected using a standard fluorescence plate reader.

The INVADER assay detects specific mutations and polymorphisms in unamplified genomic DNA. The isolated DNA sample is contacted with the first probe specific either for a polymorphism/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some embodiments, hybridization of a bound probe is detected using a TaqMan assay (PE Biosystems, Foster City, Calif., see e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

### 5. Mass Spectroscopy Assay

In some embodiments, a MassARRAY system (Sequenom, San Diego, Calif.) is used to detect polymorphisms (see e.g., U.S. Pat. No. 6,043,031). DNA is isolated from blood samples using standard procedures. Next, specific DNA regions containing the polymorphism of interest are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

### III. Kits

The invention encompasses various kits relating to compositions and methods used to identify a polymorphism of the LDLR gene present in an individual, preferably a human. In one embodiment, the kit may be used to identify a specific allele present in an individual wherein that allele includes a polymorphism in intron 11 of the LDLR gene, particularly at position 1706 minus 2 (41902 of the genomic DNA according to the NCIB database) of the LDLR gene according to SEQ ID No: 2. In another embodiment, the kit may be used to determine the genotype of an individual for both alleles.

The kit may comprise an isolated nucleic acid, preferably a primer, a set of primers, or an array of primers, as described elsewhere herein and means to contact the nucleic acids to a sample of DNA to be tested. The primers may, for example, be fixed to a solid substrate, as described elsewhere herein. The kit may further comprise a control target nucleic acid and primers. The isolated nucleic acids of the kit may also comprise a molecular label or tag. In additional embodiments, the kits of the present invention comprise various reagents necessary to practice the methods of the invention, as disclosed herein. The kit further comprises instructional material for the use thereof to be used in accordance with the teachings provided herein.

### IV. Methods of Use

The methods of the presently claimed invention can be used for a wide variety of applications including, for example, linkage and association studies, genotyping clinical populations, correlation of genotype information to phenotype information, identification and counseling of at-risk populations and pre-implantation genetic testing in assisted reproduction techniques, such as in vitro fertilization. Any analysis of genomic DNA may be benefited by a reproducible method of polymorphism analysis.

In a preferred embodiment, the methods of the presently claimed invention are used to genotype individuals, populations or samples. For example, any of the procedures described above, alone or in combination, could be used to interrogate samples obtained from a large number of individuals. Arrays may be designed and manufactured on a large scale basis to interrogate those fragments with probes comprising sequences that encompass the polymorphism at position 1706-2 (41902 of the genomic DNA) of the LDLR gene corresponding to the Arabic allele as designated herein. Thereafter, a sample from one or more individuals would be obtained and prepared using the same techniques which were used to prepare the selection probes or to design the array. Each sample can then be hybridized to an array and the hybridization pattern can be analyzed to determine the genotype of each individual or a population of individuals. Methods of use for polymorphisms and SNP discovery can be found in, for example, U.S. Pat. No. 6,361,947, which is herein incorporated by reference in its entirety for all purposes.

### Allele Frequency Determination

Large numbers of individuals, for example, 20, 40, 60, 100, 1000, 10,000, Or 100,000 or more may be genotyped at a particular SNP to determine the frequency of each of the possible alleles. Results from different populations may be compared to determine if some alleles are present at higher or lower frequencies in distinct populations. Some SNPs may be identified that are monomorphic (zero-heterozygosity) in one population but not in another population. Allele frequencies may be used to study phenomenon such as natural selection, random genetic drift, demographic evens such as population bottlenecks or expansions or combinations of these.

The present invention will be further described with reference to the following examples that illustrate the embodiments of the invention with further reference to the following drawings wherein:

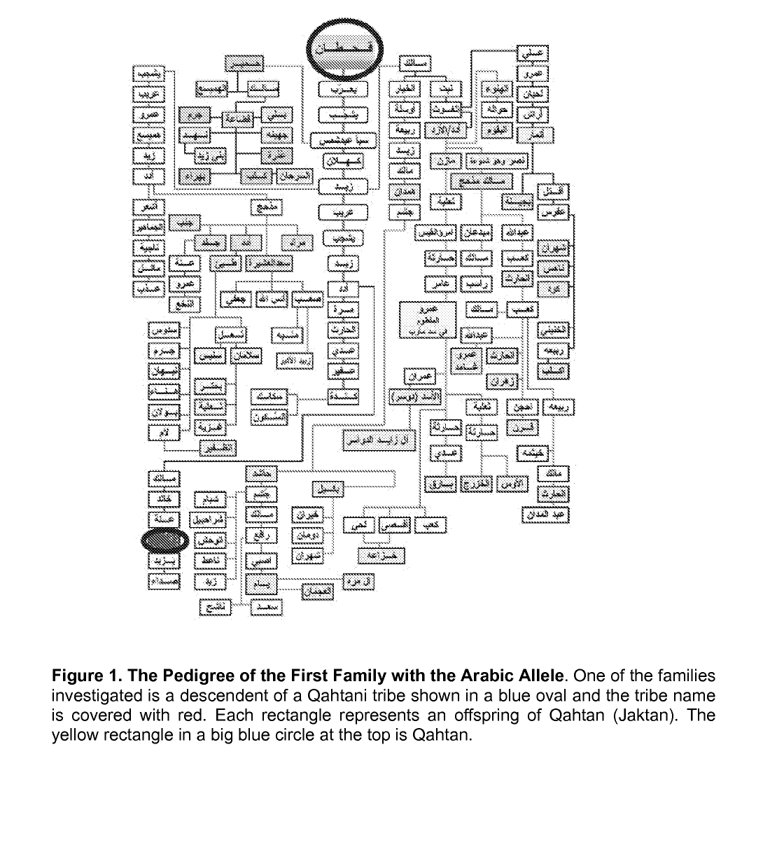

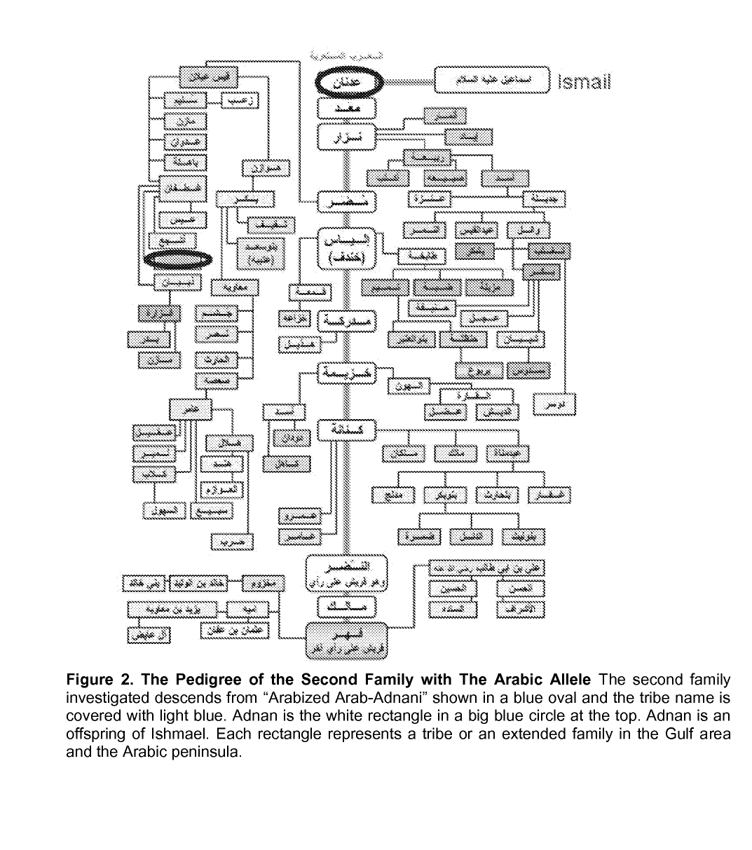

FIGS. 1 and 2 are illustrations of the pedigrees of the first and second families with the Arabic allele.

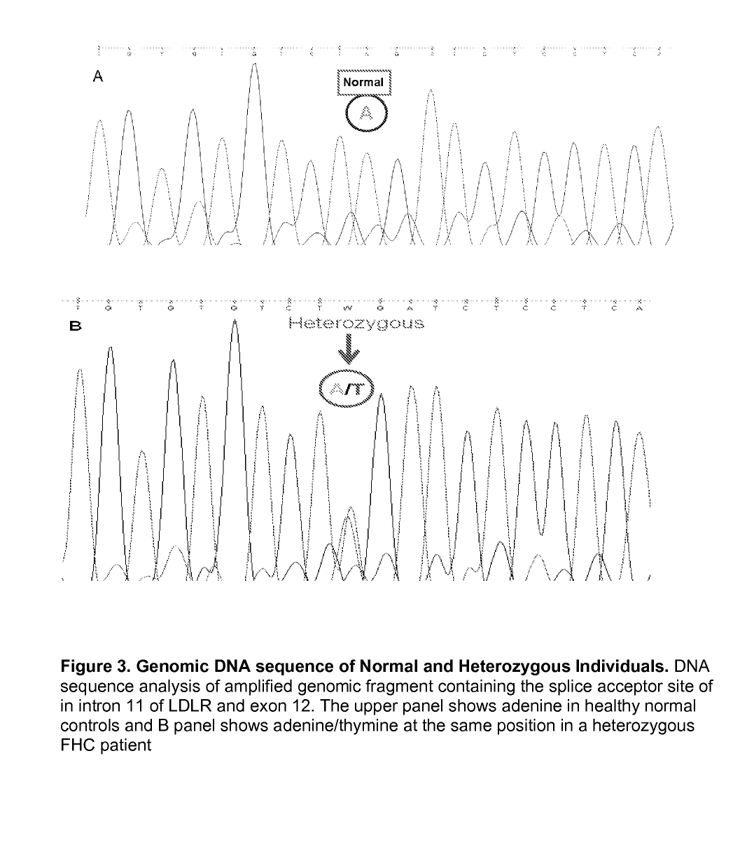

FIG. 3 is a graphic representation of genomic sequence analysis of amplified genomic fragment containing the splice acceptor site of intron 11 of LDLR and exon 12. The upper panel shows adenine in healthy normal controls and B panel shows adenine/thymine at the same position in a heterozygous FHC patient.

FIG. 4 is a genomic sequence of a homozygous individual having the mutation in intron 11 of LDLR gene.

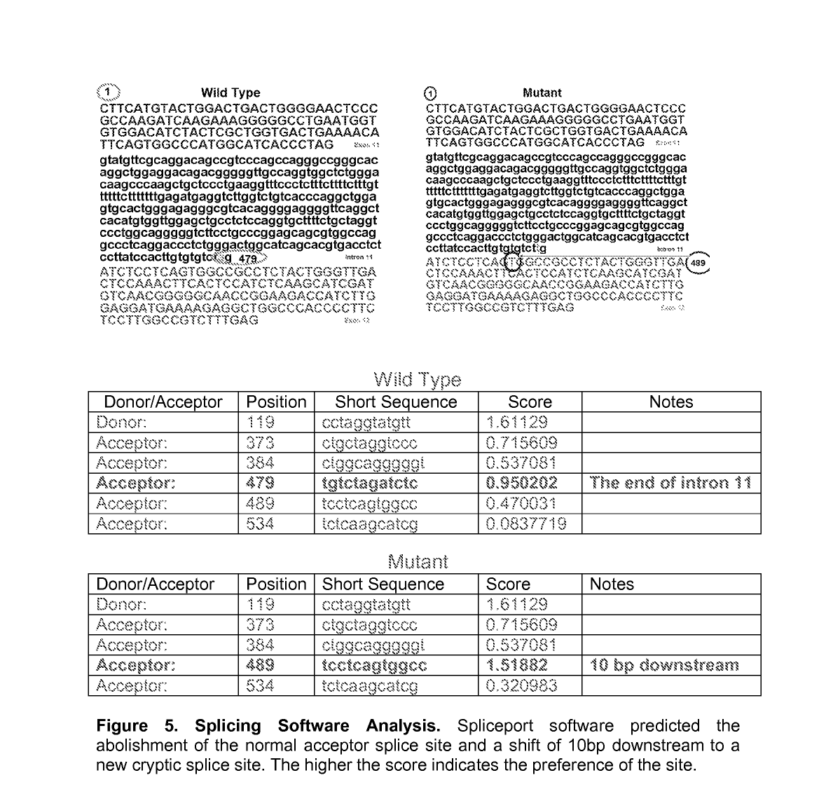

FIG. 5 is a representation of the results obtained from Splicing Software analysis predicting the abolishment of the splice acceptor site and a shift of the 10 bp downstream to a new cryptic splice site.

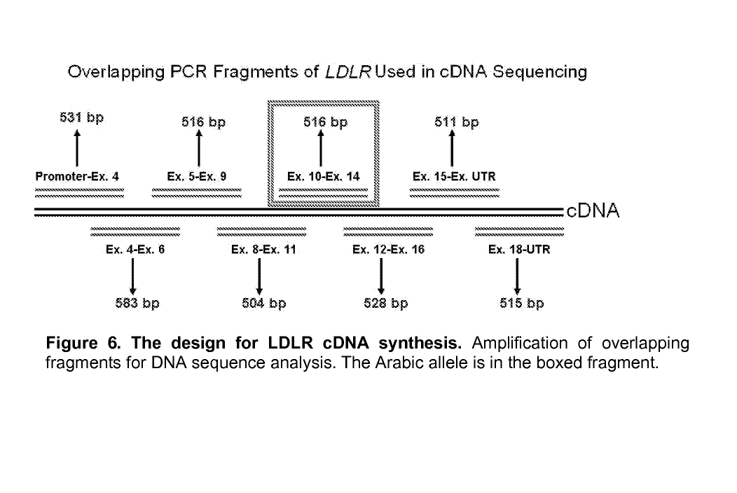

FIG. 6 is a schematic representation of the fragments used in cDNA sequencing the mutated LDLR gene.

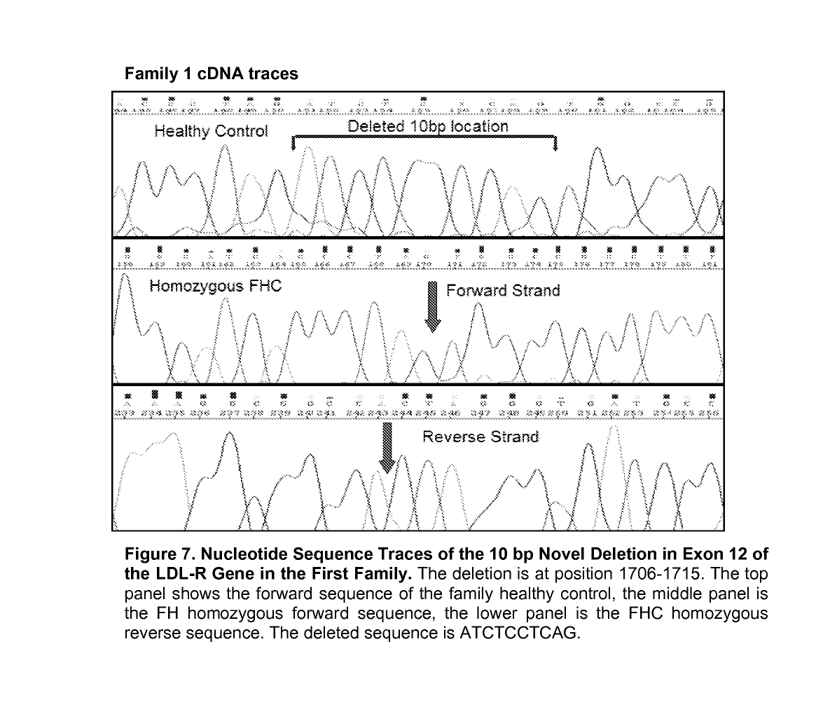

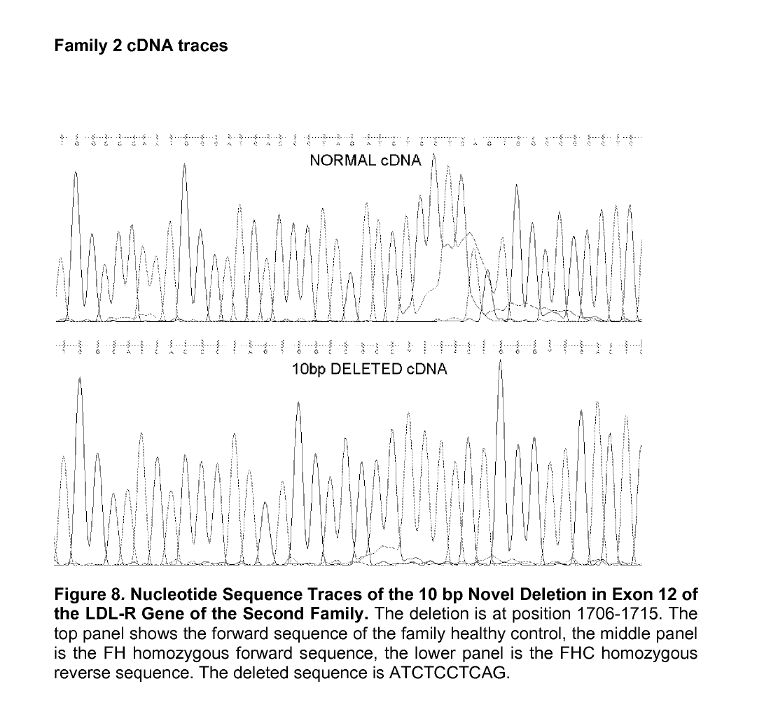

FIGS. 7 and 8 are nucleotide sequence traces of the 10 bp novel deletion in Exon 12 of the LDL-R Gene in the first and second families.

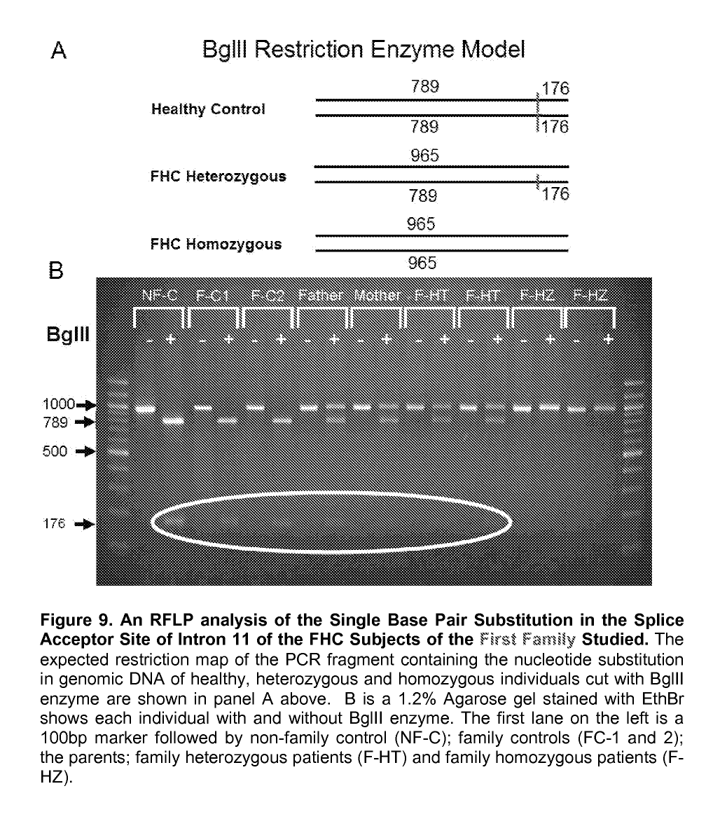

FIG. 9 is a RFLP analysis of the Single Base Pair Substitution in the Splice Acceptor Site of Intron 11 of the FHC Subjects of the First Family Studied.

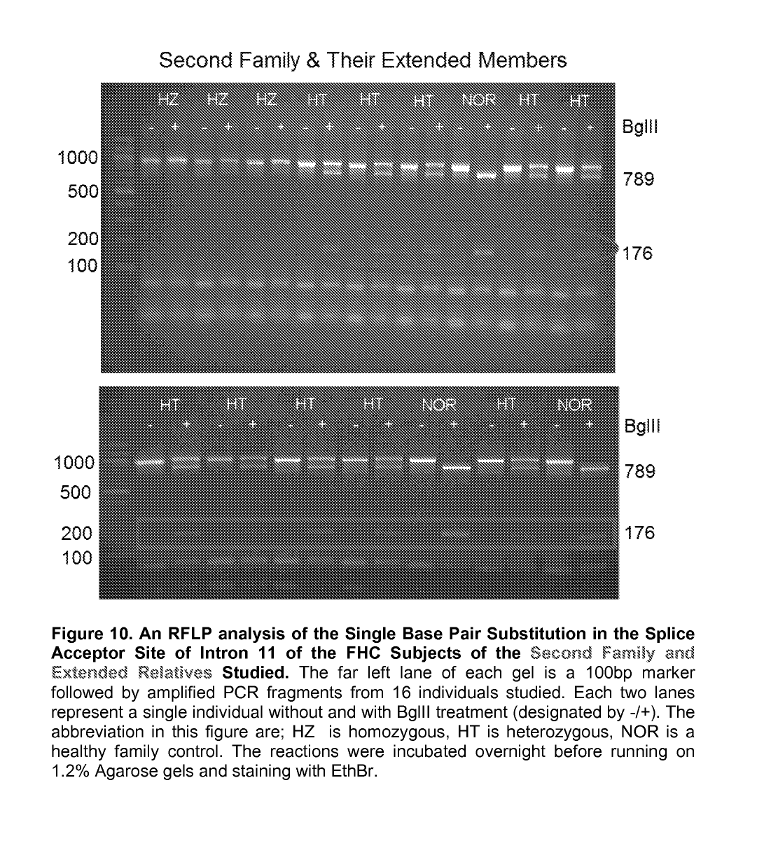

FIG. 10 is an RFLP analysis of the Single Base Pair Substitution in the Splice Acceptor Site of Intron 11 of the FHC Subjects of the Second Family and Extended Relatives Studied.

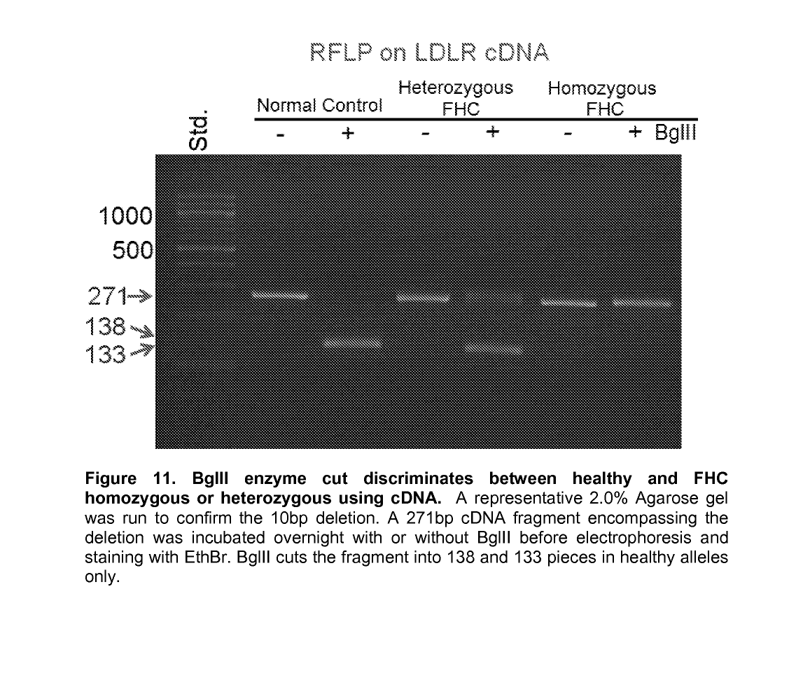

FIG. 11 is an illustration of the results obtained using BglII enzyme on the genomic DNA and which discriminates between healthy and FHC homozygous or heterozygous using cDNA.

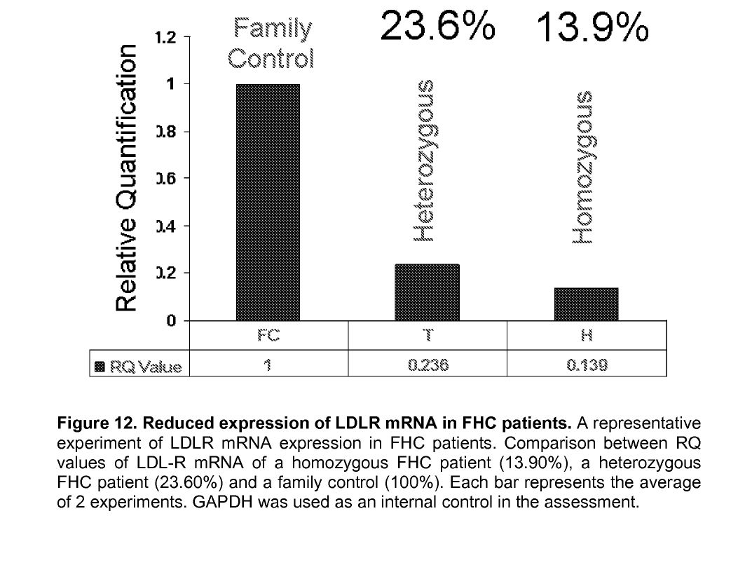

FIG. 12 is a graphic representation of the results obtained showing reduced expression of LDLR mRNA in FHC patients.

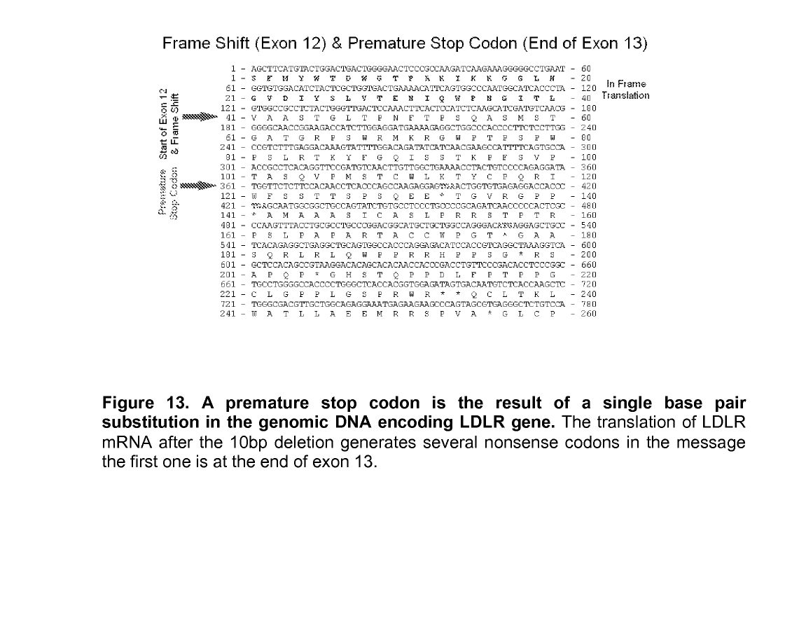

FIG. 13 illustrates the sequence in exon 12 and 13 of the LDLR gene having the substitution at position 1706-2 (41902 of the genomic DNA) and which results in a premature stop codon.

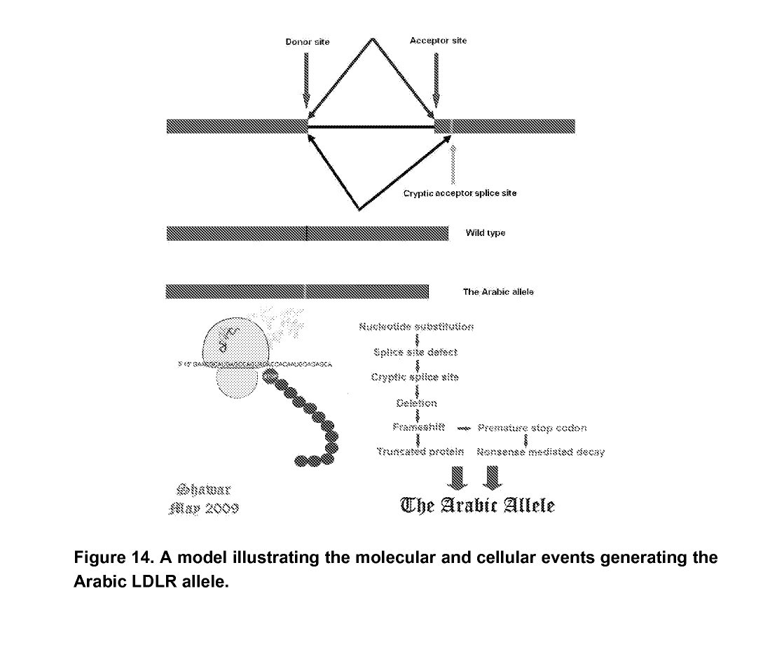

FIG. 14 is a model illustrating the molecular and cellular events generating the Arabic LDLR allele.

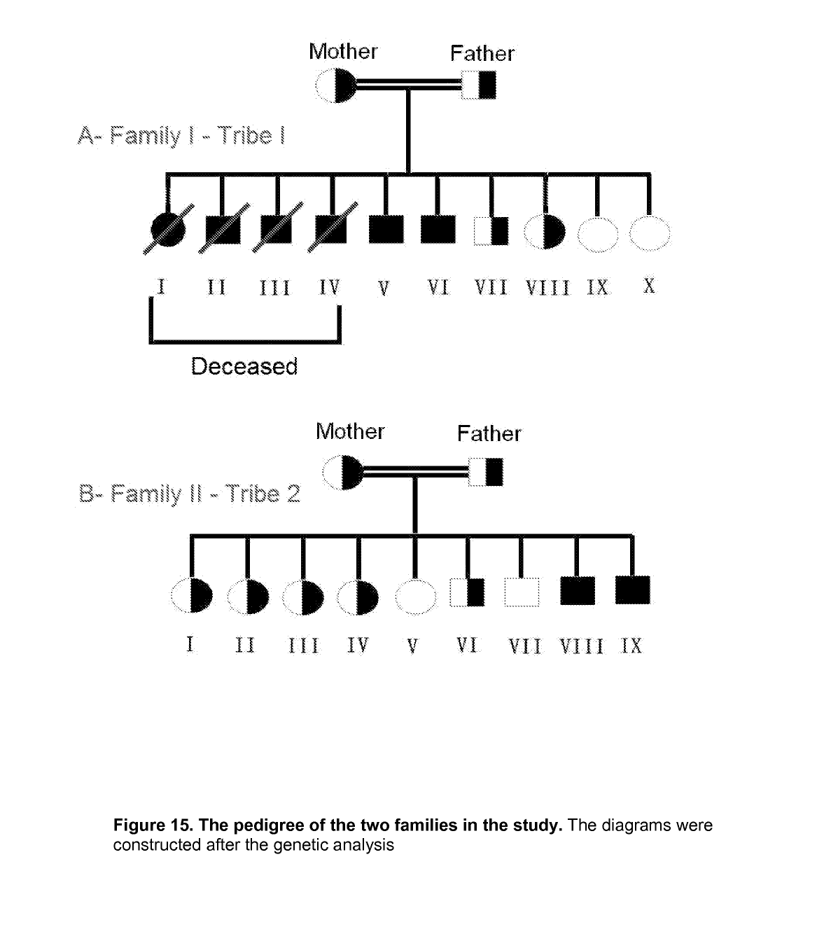

FIG. 15 is a representation of pedigree of the two families used in the study.

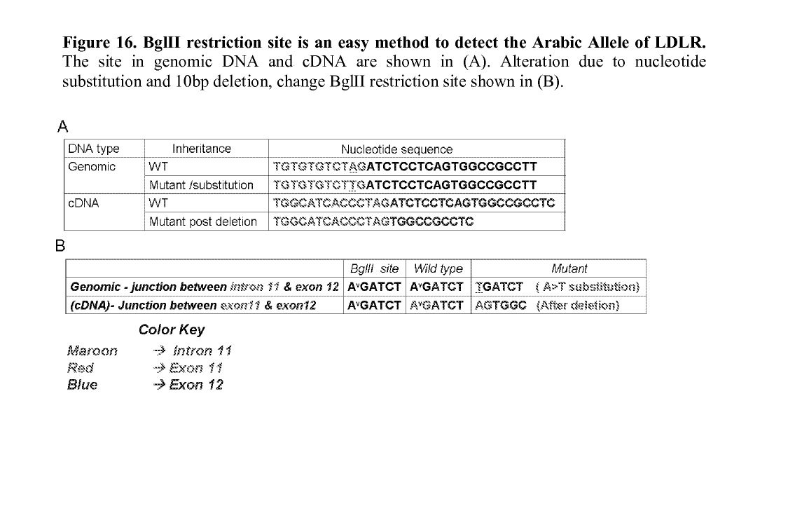

FIG. 16 is an illustration of the results obtained using BglII restriction of the Arabic allele of LDLR.

### EXAMPLE

### Study Subjects and Consents

The Institutional Ethics Committee at the Arabian Gulf University in Bahrain approved the study. The families investigated in this study were clinically diagnosed with FHC. Some of them were undergoing plasma apheresis because Statins were ineffective in their treatment. Proper consent was taken from each adult of the subjects. The consent for the minors was taken from their guardians.

### Clinical Chemistry and Clinical Diagnosis

First we selected the patients who underwent plasma apheresis at a governmental blood bank in the Gulf area. The patients' close families and extended relatives were later identified. All medical history, lipid profiles and blood chemistry were obtained after their consent.

### Blood Collection and Peripheral Blood Lymphocytes (PBLs) Isolation

Blood samples were collected in a Vacutainer containing 1.8 mg/mL K₃-EDTA. Buffy coat (PBLs) was prepared according to standard procedures described in the literature (17, 18) using Ficoll-Paque (Pharmacia, Uppsala, Sweden).

Genomic DNA Extraction from Whole Blood

Genomic DNA was isolated using QIAGEN DNA Extraction Kit according to the manufacturer instructions (QIAGEN, Valencia, Calif.). DNA concentration was determined by reading at A₂₆₀. The preparation was stored in −70° C. for subsequent use.

### RNA Extraction

We extracted RNA from whole blood samples of the volunteers using QIAamp RNA Mini Protocol Kit according to the manufacturer instructions (QIAGEN, Valencia, Calif.). The integrity of the extracted RNA was assessed by visualizing the 28S and 18S ribosomal subunits on 1.2% formaldehyde Agarose gel electrophoresis. The purity of the extracted RNA was assessed by reading at OD_{260/280} (over 1.8) and the concentration was determined by reading at A₂₆₀.

Complementary DNA (cDNA) Synthesis

Intact RNA was used to synthesize cDNA using First Strand cDNA Protoscript™ Synthesis Kit according to the instruction of the manufacturer (New England BioLabs, Ipswich, Mass.). The synthesis of GAPDH cDNA was done as an internal control to the competence of the kit. GAPDH cDNA was visualized on 1.5% Agarose gel.

### Polymerase Chain Reaction (PCR)

We used Qiagen Taq PCR Master Mix Kit (QIAGEN, Valencia, Calif.) for DNA amplification according to the manufacturer instructions. The genomic DNA or cDNA fragment(s) were used as a template with specific primers from (Thermo Electron, Waltham, Mass.) as shown in Tables 1. For genomic amplification it was optimized according to Hobbs et al., (1992) (19). Optimization for cDNA fragments' amplification was done according to Primer3 software (20) available from Massachusetts Institute of Technology, Boston, Mass. and empirically in our labs.

TABLE 1
LDLR-specific primers used to amplify the exons and the adjacent
intronic sequences from the genomic DNA of FHC patient

| Region-exon | | LDLR gene 5′-3′ | Fragment Size |
|---|---|---|---|
| Promoter | Forward | 5′-GAG TGG GAA TCA GAG CTT CAC GGG T | 155 |
| | Reverse | 5′-CCA CGT CAT TTA CAG CAT TTC AAT G | |
| Exon 1 | Forward | 5′-ACT CCT CCC CCT GCT AGA AAC CTC A | 234 |
| | Reverse | 5′-TTC TGG CGC CTG GAG CAA GCC TTA C | |
| Exon 2 | Forward | 5′-CCT TTC TCC TTT TCC TCT CTC TCA G | 172 |
| | Reverse | 5′-AAA ATA AAT GCA TAT CAT GCC CAA A | |
| Exon 3 | Forward | 5′-TGA CAG TTC AAT CCT GTC TCT TCT G | 176 |
| | Reverse | 5′-ATA GCA AAG GCA GGG CCA CAC TTA C | |
| Exon 4A | Forward | 5′-GTT GGG AGA CTT CAC ACG GTG ATG G | 355 |
| | Reverse | 5′-ACT TAG GCA GTG GAA CTC GAA GGC C | |
| Exon 4B | Forward | 5′-CCC CAG CTG TGG GCC TGC GAC AAC G | 267 |
| | Reverse | 5′-GGG GGA GCC CAG GGA CAG GTG ATA G | |
| Exon 5 | Forward | 5′-CAA CAC ACT CTG TCC TGT TTT CCA G | 173 |
| | Reverse | 5′-GGA AAA CCA GAT GGC CAG CGC TCA C | |
| Exon 6 | Forward | 5′-TCC TTC CTC TCT CTG GCT CTC ACA G | 174 |
| | Reverse | 5′-GCA AGC CGC CTG CAC CGA GAC TCA C | |
| Exon 7 | Forward | 5′-AGT CTG CAT CCC TGG CCC TGC GCA G | 169 |
| | Reverse | 5′-AGG GCT CAG TCC ACC GGG GAA TCA C | |
| Exon 8 | Forward | 5′-CCA AGC CTC TTT CTC TCT CTT CCA G | 175 |
| | Reverse | 5′-CCA CCC GCC GCC TTC CCG TGC TCA C | |
| Exon 9 | Forward | 5′-TCC ATC GAC GGG TCC CCT CTG ACC C | 271 |
| | Reverse | 5′-AGC CCT CAT CTC ACC TGC GGG CCA A | |
| Exon 10A | Forward | 5′-AGA TGA GGG CTC CTG GTG CGA TGC C | 202 |
| | Reverse | 5′-GCC CTT GGT ATC CGC AAC AGA GAC A | |
| Exon 10B | Forward | 5′-GAT CCA CAG CAA CAT CTA CTG GAC C | 162 |
| | Reverse | 5′-AGC CCT CAG CGT CGT GGA TAC GCA C | |
| Exon 11 | Forward | 5′-CAG CTA TTC TCT GTC CTC CCA CCA G | 168 |
| | Reverse | 5′-TGG GAC GGC TGT CCT GCG AAC ATA C | |
| Exon 12 | Forward | 5′-GCA CGT GAC CTC TCC TTA TCC ACT T | 209 |
| | Reverse | 5′-CAC CTA AGT GCT TCG ATC TCG TAC G | |
| Exon 13 | Forward | 5′-GTC ATC TTC CTT GCT GCC TGT TTA G | 217 |
| | Reverse | 5′-GTT TCC ACA AGG AGG TTT CAA GGT T | |
| Exon 14 | Forward | 5′-CCT GAC TCC GCT TCT TCT GCC CCA G | 202 |
| | Reverse | 5′-CGC AGA AAC AAG GCG TGT GCC ACA C | |
| Exon 15 | Forward | 5′-GAA GGG CCT GCA GGC ACG TGG CAC T | 246 |
| | Reverse | 5′-GTG TGG TGG CGG GCC CAG TCT TTA C | |
| Exon 16 | Forward | 5′-CCT CAC TCT TGC TTC TCT CCT GCA G | 127 |
| | Reverse | 5′-CGC TGG GGG ACC GGC CCG CGC TTA C | |
| Exon 17 | Forward | 5′-TGA CAG AGC GTG CCT CTC CCT ACA G | 207 |
| | Reverse | 5′-GCT TTC TAG AGA GGG TCA CAC TCA C | |
| Exon 18 | Forward | 5′-TCC GCT GTT TAC CAT TTG TTG GCA G | 135 |
| | Reverse | 5′-AAT AAA ACA AGG CCG GCG AGG TCT C | |

PCR of LDL-R Gene Fragments from cDNA

For best results in DNA sequence analysis we synthesized overlapping fragments to cover the entire cDNA. The fragments ranged in size between 504-583 bp as shown in FIG. 4 and, the primers used are shown in Table 2.

TABLE 2
LDLR-specific primers used to generate LDLR cDNA.

| | | | Fragment Size |
|---|---|---|---|
| Fragment No. | Regions/Exons | Primers Sequence | (bp) |
| 1 | Promoter-4 | F-ctaggacacagcaggtccgtg | 531 |
| | | R-ggagctgttgcactggaag | |
| 2 | 4-6 | F-acgatgggaagtgcatctct | 583 |
| | | R-ttgatgggttcatctgacca | |
| 3 | 5-9 | F-accgggaatatgactgcaag | 516 |
| | | R-tcctcaggttggggatga | |
| 4 | 8-11 | F-tggagggtggctacaagtg | 504 |
| | | R-gagttccccagtcagtccag | |
| 5 | 10-14 | F-gtctctgttgcggataccaag | 516 |
| | | R-cctctcacaccagttcactcc | |
| 6 | 12-16 | F-gccgtctttgaggacaaagtat | 528 |
| | | R-cacgctactgggcttcttct | |
| 7 | 15-UTR | F-aggacacagcacacaaccac | 511 |
| | | R-tctgcctcccagatgaataaa | |
| 8 | 18-UTR | F-tcagtctggaggatgacgtg | 515 |
| | | R-caaaggctaacctggctgtc | |

PCR of LDL-R Exon 11-12 from Total Genomic DNA

Exon 11-12, including intron 11, of the LDL-R gene was amplified from total genomic DNA using 20 pmol/μl of exon 11 forward and exon 12 reverse primers (Thermo Electron, Germany), with a fragment size of 965 bp

### Restriction Fragment Length Polymorphism (RFLP)

BglII restriction enzyme from (New England BioLabs, Ipswich, Mass.) was used to cut the genomic and cDNA PCR products. Samples from homozygous, heterozygous FHC patients, a non family healthy control and a family healthy control were incubated at 37° C. with BglII enzyme for complete digestion. The reaction mixture was incubated overnight before the enzyme was heat inactivated and the mixture was loaded on proper percentage of Agarose gel for electrophoreses. We used 100 or 50 bp DNA markers as molecular weight standards. The gel was stained with EthBr and documented by Gel-Doc 2000 Software.

### ExoSAP-IT® Treatment of PCR Products

To remove excess primers and nucleotides from the PCR products, the reaction product was treated with ExoSAP-IT according to the manufacturer instructions (USB, Cleveland, Ohio). The cleaned PCR products were ready for sequencing.

### Sequencing of LDL-R Gene

Following the ExoSAP-IT treatment of PCR fragments, DNA sequence analysis was performed using ABI Prism® BigDye® Terminator v3.1 Cycle Sequencing Kit according to the manufacturer instructions (ABI, Foster City, Calif.). Sequencing was run on an ABI3100 Genetic Analyzer automated DNA sequencer with plates of 96 wells. A 50 cm capillary was loaded with POP6 (Optimized Performance Polymer) and 10× sequencing buffer with EDTA. All samples were analyzed utilizing Applied Biosystems Sequencing Analysis Software v5.1.1.

### Flowcytometry

PBLs from FHC patients and healthy controls were cultured in regular 1640 RPMI media or in lipid deficient 1640 RPMI media (LDM). Cells were incubated with C7, LDLR-specific monoclonal antibodies (Fitzgerald, Concord, Mass.) and washed 3× before incubation with FITC-labeled secondary antibodies. Samples were fixed with 500 μl of PBS containing 1% formaldehyde, and read on flow cytometer (EPICS ALTRA-COULTER®).

### Real Time PCR

cDNA was synthesized using total RNA isolated from freshly collected PBLs. The relative expression of LDLR was measured by comparing the binding of LDLR primers to the standard binding of GAPDH primers. A duplicate mixture of 25 μl in volume for each case, including the internal negative control (RNase-free water was used instead of cDNA). RT-PCR was measured using (TaqMan® Gene Expression Assays, Applied Biosystems, USA) containing blue-colored FAM and non-fluorescenated quencher; as well as, 20× internal control human GAPDH probe mixture (Pre-Developed TaqMan® Assay Reagents, Applied Biosystems) that consists of GAPDH forward and reverse primers, and GAPDH probes containing green-colored VIC fluorescent reporter dye and TAMRA Quencher.

### Results

### History and Genealogy

The individuals investigated here are from two unrelated families (tribes) according to a combination of Arab historians, Judo-Christian traditions and genealogists. One tribe is a descendant of Qahtani Arabs (Jaktan, Genesis 10:25-26) and the second is a descendent of Adnani Arabs (Arabized Arabs) who are accepted to have descended from Adnan, an offspring of Ishmael. The tribes of Qahtani and Adnani Arabs are shown in FIGS. 1 and 2 respectively (21, 22, 23, 24). Each tribe is spread in the Gulf area and the Arab peninsula.

Clinical Diagnosis of the Individuals in this Study

The two families were clinically diagnosed with FHC according different clinical diagnostic criteria. Total cholesterol level, medical history, current treatment, and clinical symptoms of the individuals studied from two separate families are shown in Tables 3 and 4 and in FIG. 15. We studied more relatives of the second family (living in a different Gulf country). Our results indicate that they have the similar clinical chemistry results and mutation (data not shown).

TABLE 3
Clinical Chemistry of the First Family. Age, cholesterol level, medical
and medication history and FHC clinical Of first family members.

| | | | | | | Cholesterol |
|---|---|---|---|---|---|---|
| Family | Pedigree | | | FHC According to | | Level |
| Member | Designation | Age Yrs | Clinical Symptoms | Clinical Criteria | Other Remarks | (mmol/L) |
| Father | Father | 51 | Coronary Heart | Heterozygous | Under medication | 8.9 |
| | | | Disease | | of Lovastatin | |
| | | | Hypertension | | | |
| | | | (180/118) | | | |
| Mother | Mother | 46 | — | Heterozygous | Under medication | 9.8 |
| | | | | | of Lovastatin | |
| Daughter | I | 4 | Atherosclerosis | Homozygous | — | Deceased |
| Son | II | 10 | Atherosclerosis | Homozygous | — | Deceased |
| Son | III | 11 | Atherosclerosis | Homozygous | — | Deceased |
| Son | IV | 12 | Atherosclerosis | Homozygous | — | Deceased |
| Son | V | 16 | Joints Xanthomas | Homozygous | Xanthomas disappeared | >18.0 before |
| | | | since 9 yrs | | after ~100 apheresis | apheresis |
| | | | | | | ~10.0 during |
| | | | | | | apheresis |
| Son | VI | 18 | Joints Xanthomas | Homozygous | Xanthomas disappeared | >18.0 before |
| | | | since 8 yrs | | after ~100 apheresis | apheresis |
| | | | | | | ~10.0 during |
| | | | | | | apheresis |
| Son | VII | 10 | — | Heterozygous | Under medication of | 8.9 |
| | | | | | Lovastatin | |
| Daughter | VIII | 29 | — | Heterozygous | Pregnant at the time | 12.8 |
| | | | | | of cholesterol | |
| | | | | | measurement | |
| Daughter | IX | 14 | — | Normal | — | Normal Level |
| Daughter | X | 26 | — | Normal | — | Normal Level |

Note:
Normal level of blood cholesterol is 3.0-5.2 mmol/L. Blood was taken from patients after overnight fasting for 12-14 hours.

TABLE 4
Clinical Chemistry of the Second Family. Age, cholesterol level, medical and medication
history and FHC clinical diagnosis of second family member investigated.

| | | | | | | Cholesterol |
|---|---|---|---|---|---|---|
| Family | Pedigree | | Clinical | FHC by Clinical | Lipitore | Level |
| Member | Designation | Age (yrs) | Symptoms | Criteria | Medication | (mmol/L) |
| Father | Father | 53 | CHD diabetic | Heterozygous | Yes | 8.9 |
| Mother | Mother | 42 | — | Heterozygous | Yes | 5.5 |
| Daughter | I | 11 | — | Heterozygous | Yes | 8.9 |
| Daughter | II | 18 | — | Heterozygous | Yes | 5.9 |
| Daughter | III | 19 | — | Heterozygous | Yes | 8.1 |
| Daughter | IV | 23 | Diabetic takes, | Heterozygous | Yes | 10.2 |
| | | | insulin injection | | | |
| Daughter | V | 22 | — | Normal | No | 4.7 |
| Son | VI | 9 | Has joints | Homozygous | Statins resistant, | 25-35 before |
| | | | Xanthomas before | | gets apheresis | apheresis |
| | | | 7 yrs. Disappeared | | weekly | and ~14 after |
| | | | after many aphaeresis | | | the procedures |
| | | | procedures. | | | |
| Son | VII | 14 | Has joints | Homozygous | Statins resistant, | 25-35 before |
| | | | Xanthomas before | | gets apheresis | apheresis |
| | | | 9 yrs, Disappeared | | weekly | and ~14 after |
| | | | after many aphaeresis | | | the procedures |
| | | | procedures. | | | |
| Son | VIII | 10 | — | Heterozygous | — | 6.0 |
| Son | IX | 17 | Normal | Normal | — | 3.7 |

Note:
Normal level of blood cholesterol is 3.0-5.2 mmol/L. Blood was taken from patients after overnight fasting for 12-14 hours.

### A Novel Substitution in the Acceptor Splice Site of LDLR Intron 11

Genomic DNA from different individuals of an Arab family clinically diagnosed with FHC was amplified using specific primers shown in Table 1. The primers cover LDLR exons and the adjacent intronic sequences. DNA sequence analysis of the amplified PCR fragments revealed a single nucleotide substitution at position 1706-2 (41902 of the genomic DNA according to the NCIB database) in the acceptor splice site of intron 11 in the LDLR (1706-2, A>T) as shown in FIGS. 3 and 4. The substitution was detected in the homozygous and heterozygous FHC individuals. Non-family healthy control and a family healthy analyzed along side showed the normal adenine nucleotide. The mutation is novel and has not been described before in the literature or on in the LDLR data base on the website of University College London as shown in Table 5. http://www.ucl.ac.uk/ldlr/Current/index.php?select_db=LDLR

TABLE 5
The University College London Database for LDLR. The only mutations
in the splice site junction of intron 11 and exon 12 in the databases
of the University College London are shown below. These mutations
were found in Japan (JP) and in Great Britain (GB).

| | | | | | |
|---|---|---|---|---|---|
| 11 | 1705 + | Hattori et al 2002 J | LDLR_00997 | 5′ splice | JP |
| | 1G > C | Hum Genet 47 80 | | donor | |
| | | | | mutation in | |
| | | | | intron 11 | |
| 12 | 1706 − | Graham et al 2005 | LDLR_01159 | 3′ splice | GB |
| | 1G > A | Atherosclerosis 182 | | acceptor | |
| | | 331 | | mutation in | |
| | | | | intron 11. | |

### In Silico Prediction of the Novel Substation Effect

Using mRNA sequence encompassing exon 11, intron 11 and exon 12 (FIG. 5), we evaluated the effect of this substitution on splicing (in silico) using Spliceport software available from the University of Maryland, http://spliceport.cs.umd.edu/SplicingAnalyser2.html. As shown in FIG. 5. Spliceport predicted the abolishment of the normal splice site in position 479 as shown in top panel of Figure. The creation of a new cryptic acceptor splice site was with high score (FIG. 5, lower panel). The new site is 10 bp downstream of the normal splice site in the pre-mRNA. If, the new splice site is used, it is predicted that a 10 bp deletion, a frame shift and a premature stop codon will follow. The model in FIG. 14, illustrates the predicted events due to the substitution. Normal donor and acceptor splice sites of LDLR are shown in Table 6 below

TABLE 6
Splice donor and acceptor sites of
the 17 introns in LDLR

| | Donor | Acceptor | |
|---|---|---|---|
| Intron | 100% → 5′GT | 100% → 5′AG | Notes |
| 1 | gtaagg | ctcag | |
| 2 | gtgag | ctgtag | |
| 3 | gtaagt | ctgcag | |
| 4 | gtatgg | ttccag | |
| 5 | gtgagc | tcacag | |
| 6 | gtgagt | gcgcag | |
| 7 | gtgatt | ttccag | |
| 8 | gtgagc | ccccag | |
| 9 | gtgagc | cctcag | |
| 10 | gtgcgt | caccag | |
| 11 | gtatg | gtctag | 360 bp |
| 12 | gtgtgg | gtttag | |
| 13 | gtaaggg | ccccag | |
| 14 | gtgtgg | tttcag | |
| 15 | gtaaag | ctgcag | |
| 16 | gtaagc | ctacag | |
| 17 | gtgagt | tggcag | |

### Wet Lab Investigation

To test the in silico results, we generated complementary DNA (cDNA) from the patients and family and non-family controls by RT-PCR. Overlapping PCR fragments were designed to cover the whole LDLR cDNA as shown in FIG. 4. Fragment 5 covers the substitution and the predicted deletion area. DNA sequence analysis shows a ten base pair deletion from exon 5 confirming our in silico predictions. The sequence is shown in FIG. 5. Thus, and wet lab confirm the presence of a cryptic splice site

### RFLP

BglII restriction enzyme cuts between adenine and guanine nucleotides in the hexa-nucleotides 5′ A^{V}GATCT3′. The substitution in position 1706-2 or 41902 of the genomic DNA and the 10 bp deletion in the cDNA alter the normal site of the restriction enzyme as shown in FIG. 16. Thus, BglII fails to cut the mutant form of the gene as shown in FIGS. 9 and 10 (family 1 and 2 respectively (and their extended family) and FIG. 11 (cDNA).

LDLR Expression in FHC patients

The expression of LDLR protein and mRNA from the FHC individuals studied and healthy normal controls was assessed by flowcytometry and real-time PCR respectively.

### Flowcytometry

Using C7, an LDLR-specific monoclonal antibody was used in these experiments to assess the surface expression on PBLs taken from FHC patients or healthy individuals. The cells were incubated in regular medium or LDM to induce the expression of the receptor. Both the homozygous and heterozygous FHC individuals show low expression as shown in Table 7.

TABLE 7
Reduced expression of LDLR on the surface of PBLs from FHC patients.
A representative experiment showing relative quantification of C7
monoclonal bound to LDLR on PBLs from FHC patients and healthy control.
Each reading is the average of two tubes. Stimulation index is the
median fluorescence (MF) of cells grown in lipid deficient serum −
MF of cells grown in regular 1640 RPMI.

| | Subjects | | | | | |
|---|---|---|---|---|---|---|
| | Non-Family control | | Heterozygous | | Homozygous | |
| Mean fluorescence | RPMI | LPDS | RPMI | LPDS | RPMI | LPDS |
| Sample—control | 0.2 | 19.0 | 1.2 | 3.9 | 3.7 | 5.9 |

| | | | |
|---|---|---|---|
| Stimulation Index | 18.8 | 2.7 | 2.2 |
| LPDS − RPMI | | | |

### Real Time PCR

The level of LDLR transcript was extremely low in all FHC patients. The transcript ranged between 24% (heterozygous) and 14% (homozygous) compared to a family control. The transcript range correlated nicely with the protein level.

### REFERENCES

1. Watts G F, Lewis B, Sullivan D R: Familial hypercholesterolemia: a missed opportunity in preventive medicine. Nat Clin Pract Cardiovasc Med 2007; 4: 404-405.
2. Soutar A K, Naoumova R P: Mechanisms of Disease: genetic causes of familial hypercholesterolemia. Nat Clin Pract Cardiovasc Med 2007; 4: 214-225.
3. Jeon H, Blacklow S C: Structure and physiologic function of the low-density lipoprotein receptor. Annu Rev Biochem 2005; 74: 535-562.
4. Knudson A G, Jr.: Founder effect in Tay-Sachs disease. Am J Hum Genet. 1973; 25: 108.
5. Ferla R, Calo V, Cascio S et al: Founder mutations in BRCA1 and BRCA2 genes. Ann Oncol 2007; 18 Suppl 6: vi93-98.
6. Moskowitz S M, Chmiel J F, Sternen D L, Cheng E, Gibson R L, Marshall S G, Cutting G R: Clinical practice and genetic counseling for cystic fibrosis and CFTR-related disorders. Genet Med 2008, 10(12):851-868.
7. Zeegers M P, van Poppel F, Vlietinck R, Spruijt L, Ostrer H: Founder mutations among the Dutch. Eur J Hum Genet. 2004; 12: 591-600.
8. Zlotogora J: Multiple mutations responsible for frequent genetic diseases in isolated populations. Eur J Hum Genet. 2007; 15: 272-278.
9. Lieberman L, Kirby M, Ozolins L, Mosko J, Friedman J: Initial presentation of unscreened children with sickle cell disease: The Toronto experience. Pediatr Blood Cancer 2009; (published ahead online)
10. Craig I H: Make early diagnosis, prevent early death from familial hypercholesterolaemia. The MED-PED FH program. Med J Aust 1995; 162: 454-455.
11. Austin M A, Hutter C M, Zimmern R L, Humphries SE: Familial hypercholesterolemia and coronary heart disease: a HuGE association review. Am J Epidemiol 2004; 160: 421-429.
12. http://www.medped.org/
13. http://www.ucl.ac.uk/ldlr/Current/index.php?select_db=LDLR
14. Al-Gazali L, Hamamy H, Al-Arrayad S: Genetic disorders in the Arab world. Bmj 2006; 333: 831-834.
15. Fredrick E. Greenspahn, Encyclopedia of Religion, Ishmael, p. 4551-4552.
16. Van Aalst-Cohen E S, Jansen A C M, Tanck M W, Defesche J C, Trip M D, Lansberg P J, Anton F. H. Stalenhoef A F, Kastelein J J: Diagnosing familial hypercholesterolemia: the relevance of genetic testing. Eur Heart J. 2006; 27: 2240-2246.
17. Boyum A: Isolation of leucocytes from human blood. A two-phase system for removal of red cells with methylcellulose as erythrocyte-aggregating agent. Scand J Clin Lab Invest Suppl 1968; 97: 9-29.
18. Boyum A: Isolation of leucocytes from human blood. Further observations. Methylcellulose, dextran, and ficoll as erythrocyteaggregating agents. Scand J Clin Lab Invest Suppl 1968; 97: 31-50.
19. Hobbs H H, Brown M S, Goldstein J L: Molecular genetics of the LDL receptor gene in familial hypercholesterolemia. Hum Mutat 1992; 1: 445-466.
20. http://frodo.wi.mit.edu/
21. http://nabataea.net/12tribes.html
22. http://nabataea.net/arabia.html
23. http://www.newworldencyclopedia.org/entry/Arabs
24. http://www.newworldencyclopedia.org/entry/Ishmael
25. http://www.ucl.ac.uk/ldlr/Current/index.php?select_db=LDLR
26. http://spliceport.cs.umd.edu/SplicingAnalyser2.html
27. Amrani N, Dong S, He F, Ganesan R, Ghosh S, Kervestin S, Li C, Mangus D A, Spatrick P, Jacobson A: Aberrant termination triggers nonsense-mediated mRNA decay. Biochem Soc Trans 2006, 34(Pt 1):39-42.
28. Holla O L, Kulseth M A, Berge K E, Leren T P, Ranheim T: Nonsense-mediated decay of human LDL receptor mRNA. Scand J Clin Lab Invest 2009, 69(3):409-417.
29. Nishikawa S, Brodsky J L, Nakatsukasa K: Roles of molecular chaperones in endoplasmic reticulum (ER) quality control and ER-associated degradation (ERAD). J Biochem 2005, 137(5):551-555.
30. Brodsky J L: The protective and destructive roles played by molecular chaperones during ERAD (endoplasmic-reticulum-associated degradation). Biochem J 2007, 404(3):353-363.

SEQUENCE LISTING

```
GCCTGAGCCTGGCTGTTTCTTCCAGAATTCGTTGCACGCATTGGCTGGGATCCTCCCCCG
          41110 41120 41130 41140 41150 41160
CCCTCCAGCCTCACAGCTATTCTCTGTCCTCCCACCAGCTTCATGTACTGGACTGACTGG
          41170 41180 41190 41200 41210 41220
GGAACTCCCGCCAAGATCAAGAAAGGGGGCCTGAATGGTGTGGACATCTACTCGCTGGTG 11
          41230 41240 41250 41260 41270 41280
ACTGAAAACATTCAGTGGCCCAATGGCATCACCCTAGGTATGTTCGCAGGACAGCCGTCC
          41290 41300 41310 41320 41330 41340
CAGCCAGGGCCGGGCACAGGCTGGAGGACAGACGGGGGTTGCCAGGTGGCTCTGGGACAA
          41350 41360 41370 41380 41390 41400
GCCCAAGCTGCTCCCTGAAGGTTTCCCTCTTTCTTTTCTTTGTTTTTTCTTTTTTTGAGA
          41410 41420 41430 41440 41450 41460
TGAGGTCTTGGTCTGTCACCCAGGCTGGAGTGCACTGGCGCAATCGTAGCTCACTGCAGC
          41470 41480 41490 41500 41510 41520
CTCCACCTCCCAGGCTCAAGTGATCCTCCTGCCTCACCCTCCTGAGTAGCTGAGATTACA
          41530 41540 41550 41560 41570 41580
GACACGTGCCACCACGGCAGACTAATTTTATTTTATTTTTGGGAAGAGACAAAGTCTTGT
          41590 41600 41610 41620 41630 41640
TATGTTGGCCTGGCTGGTCTCAAACTCAGGGTGCAAGCGATCCTCCCGCCTCAGCCTTCC
          41650 41660 41670 41680 41690 41700
AAACTGCTGGGATTACAGGCGTGGGCCACCGTACCCAGCCTCCTTGAAGTTTTTCTGACC
          41710 41720 41730 41740 41750 41760
TGCAACTCCCCTACCTGCCCATTGGAGAGGGCGTCACAGGGGAGGGGTTCAGGCTCACAT
          41770 41780 41790 41800 41810 41820
GTGGTTGGAGCTGCCTCTCCAGGTGCTTTTCTGCTAGGTCCCTGGCAGGGGGTCTTCCTG
          41830 41840 41850 41860 41870 41880
CCCGGAGCAGCGTGGCCAGGCCCTCAGGACCCTCTGGGACTGGCATCAGCACGTGACCTC
          41890 41900 41910 41920 41930 41940
TCCTTATCCACTTGTGTGTCTAGATCTCCTCAGTGGCCGCCTCTACTGGGTTGACTCCAA
          41950 41960 41970 41980 41990 42000
ACTTCACTCCATCTCAAGCATCGATGTCAACGGGGGCAACCGGAAGACCATCTTGGAGGA 12
          42010 42020 42030 42040 42050 42060
TGAAAAGAGGCTGGCCCACCCCTTCTCCTTGGCCGTCTTTGAGGTGTGGCTTACGTACGA
          42070 42080 42090 42100 42110 42120
GATGCAAGCACTTAGGTGGCGGATAGACACAGACTATAGATCACTCAAGCCAAGATGAAC
          42130 42140 42150 42160 42170 42180
GCAGAAAACTGGTTGTGACTAGGAGGAGGTCTTAGACCTGAGTTATTTCTATTTTCTTCT
          42190 42200 42210 42220 42230 42240
SEQ ID NO 1, the wild type sequence of human LDLR gene, a partial segment
encompassing exon 11, IVS12 and exon 12. Adenine in position 41902 is the
natural nucleotide (shown in red).
GCCTGAGCCTGGCTGTTTCTTCCAGAATTCGTTGCACGCATTGGCTGGGATCCTCCCCCG
          41110 41120 41130 41140 41150 41160
CCCTCCAGCCTCACAGCTATTCTCTGTCCTCCCACCAGCTTCATGTACTGGACTGACTGG
          41170 41180 41190 41200 41210 41220
GGAACTCCCGCCAAGATCAAGAAAGGGGGCCTGAATGGTGTGGACATCTACTCGCTGGTG 11
          41230 41240 41250 41260 41270 41280
ACTGAAAACATTCAGTGGCCCAATGGCATCACCCTAGGTATGTTCGCAGGACAGCCGTCC
          41290 41300 41310 41320 41330 41340
CAGCCAGGGCCGGGCACAGGCTGGAGGACAGACGGGGGTTGCCAGGTGGCTCTGGGACAA
          41350 41360 41370 41380 41390 41400
GCCCAAGCTGCTCCCTGAAGGTTTCCCTCTTTCTTTTCTTTGTTTTTTCTTTTTTTGAGA
          41410 41420 41430 41440 41450 41460
TGAGGTCTTGGTCTGTCACCCAGGCTGGAGTGCACTGGCGCAATCGTAGCTCACTGCAGC
          41470 41480 41490 41500 41510 41520
CTCCACCTCCCAGGCTCAAGTGATCCTCCTGCCTCACCCTCCTGAGTAGCTGAGATTACA
          41530 41540 41550 41560 41570 41580
GACACGTGCCACCACGGCAGACTAATTTTATTTTATTTTTGGGAAGAGACAAAGTCTTGT
          41590 41600 41610 41620 41630 41640
TATGTTGGCCTGGCTGGTCTCAAACTCAGGGTGCAAGCGATCCTCCCGCCTCAGCCTTCC
          41650 41660 41670 41680 41690 41700
AAACTGCTGGGATTACAGGCGTGGGCCACCGTACCCAGCCTCCTTGAAGTTTTTCTGACC
          41710 41720 41730 41740 41750 41760
TGCAACTCCCCTACCTGCCCATTGGAGAGGGCGTCACAGGGGAGGGGTTCAGGCTCACAT
          41770 41780 41790 41800 41810 41820
GTGGTTGGAGCTGCCTCTCCAGGTGCTTTTCTGCTAGGTCCCTGGCAGGGGGTCTTCCTG
          41830 41840 41850 41860 41870 41880
CCCGGAGCAGCGTGGCCAGGCCCTCAGGACCCTCTGGGACTGGCATCAGCACGTGACCTC
          41890 41900 41910 41920 41930 41940
TCCTTATCCACTTGTGTGTCTTGATCTCCTCAGTGGCCGCCTCTACTGGGTTGACTCCAA
          41950 41960 41970 41980 41990 42000
ACTTCACTCCATCTCAAGCATCGATGTCAACGGGGGCAACCGGAAGACCATCTTGGAGGA 12
          42010 42020 42030 42040 42050 42060
TGAAAAGAGGCTGGCCCACCCCTTCTCCTTGGCCGTCTTTGAGGTGTGGCTTACGTACGA
          42070 42080 42090 42100 42110 42120
GATGCAAGCACTTAGGTGGCGGATAGACACAGACTATAGATCACTCAAGCCAAGATGAAC
          42130 42140 42150 42160 42170 42180
GCAGAAAACTGGTTGTGACTAGGAGGAGGTCTTAGACCTGAGTTATTTCTATTTTCTTCT
          42190 42200 42210 42220 42230 42240
SEQ ID NO 2: Partial sequence of LDLR genomic DNA, the sequence covers the
segment between exon 11, IVS 11 and exon 12.
Exon 1
1 31
ATG GGG CCC TGG GGC TGG AAA TTG CGC TGG ACC GTC GCC TTG CTC CTC GCC GCG GCG GGG
Met gly pro trp gly trp lys leu arg trp thr val ala leu leu leu ala ala ala gly
−21/1 −11/11
Exon 2
61 68 91
ACT GCA GTG GGC GAC AGA TGT GAA AGA AAC GAG TTC CAG TGC CAA GAC GGG AAA TGC ATC
thr ala val gly asp arg cys glu arg asn glu phe gln cys gln asp gly lys cys ile
−1/21 10/31
121 151
TCC TAC AAG TGG GTC TGC GAT GGC AGC GCT GAG TGC CAG GAT GGC TCT GAT GAG TCC CAG
ser tyr lys trp val cys asp gly ser ala glu cys gln asp gly ser asp glu ser gln
20/41 30/51
Exon 3
181 191 211
GAG ACG TGC TTG TCT GTC ACC TGC AAA TCC GGG GAC TTC AGC TGT GGG GGC CGT GTC AAC
glu thr cys ler ser val thr cys lys ser gly asp phe ser cys gly gly arg val asn
40/61 50/71
241 271
CGC TGC ATT CCT CAG TTC TGG AGG TGC GAT GGC CAA GTG GAC TGC GAC AAC GGC TCA GAC
arg cys ile pro gln phe trp arg cys asp gly gln val asp cys asp asn gly ser asp
60/81 70/91
Exon 4
301 314 331
GAG CAA GGC TGT CCC CCC AAG ACG TGC TCC CAG GAC GAG TTT CGC TGC CAC GAT GGG AAG
glu gln gly cys pro pro lys thr cys ser gln asp glu phe arg cys his asp gly lys
80/101 90/111
361 391
TGC ATC TCT CGG CAG TTC GTC TGT GAC TCA GAC CGG GAC TGC TTG GAC GGC TCA GAC GAG
cys ile ser arg gln phe val cys asp ser asp arg asp cys leu asp gly ser asp glu
100/121 110/131
421 451
GCC TCC TGC CCG GTG CTC ACC TGT GGT CCC GCC AGC TTC CAG TGC AAC AGC TCC ACC TGC
ala ser cys pro val leu thr cys gly pro ala ser phe gln cys asn ser ser thr cys
120/141 130/151
481 511
ATC CCC CAG CTG TGG GCC TGC GAC AAC GAC CCC GAC TGC GAA GAT GGC TCG GAT GAG TGG
ile pro gln leu trp ala cys asp asn asp pro asp cys glu asp gly ser asp glu trp
140/161 150/171
541 571
CCG CAG CGC TGT AGG GGT CTT TAC GTG TTC CAA GGG GAC AGT AGC CCC TGC TCG GCC TTC
pro gln arg cys arg gly leu tyr val phe gln gly asp ser ser pro cys ser ala phe
160/181 170/191
601 631
GAG TTC CAC TGC CTA AGT GGC GAG TGC ATC CAC TCC AGC TGG CGC TGT GAT GGT GGC CCC
glu phe his cys leu ser gly glu cys ile his ser ser trp arg cys asp gly gly pro
180/201 190/211
Exon 5
661 691 695
GAC TGC AAG GAC AAA TCT GAC GAG GAA AAC TGC GCT GTG GCC ACC TGT CGC CCT GAC GAA
asp cys lys asp lys ser asp glu glu asn cys ala val ala thr cys arg pro asp glu
200/221 210/231
721 751
TTC CAG TGC TCT GAT GGA AAC TGC ATC CAT GGC AGC CGG CAG TGT GAC CGG GAA TAT GAC
phe gln cys ser asp gly asn cys ile his gly ser arg gln cys asp arg glu tyr asp
220/241 230/251
Exon 6
781 811 818
TGC AAG GAC ATG AGC GAT GAA GTT GGC TGC GTT AAT GTG ACA CTC TGC GAG GGA CCC AAC
cys lys asp met ser asp glu val gly cys val asn val thr leu cys glu gly pro asn
240/261 250/271
841 871
AAG TTC AAG TGT CAC AGC GGC GAA TGC ATC ACC CTG GAC AAA GTC TGC AAC ATG GCT AGA
lys phe lys cys his ser gly glu cys ile thr leu asp lys val cys asn met ala arg
260/281 270/291
Exon 7
901 931 941
GAC TGC CGG GAC TGG TCA GAT GAA CCC ATC AAA GAG TGC GGG ACC AAC GAA TGC TTG GAC
asp cys arg asp trp ser asp glu pro ile lys glu cys gly thr asn glu cys leu asp
280/301 290/311
961 991
AAC AAC GGC GGC TGT TCC CAC GTC TGC AAT GAC CTT AAG ATC GGC TAC GAG TGC CTG TGC
asn asn gly gly cys ser his val cys asn asp leu lys ile gly tyr glu cys leu cys
300/321 310/331
Exon 8
1021 1051 1061
CCC GAC GGC TTC CAG CTG GTG GCC CAG CGA AGA TGC GAA GAT ATC GAT GAG TGT CAG GAT
pro asp gly phe gln leu val ala gln arg arg cys glu asp ile asp glu cys gln asp
320/341 330/351
1081 1111
CCC GAC ACC TGC AGC CAG CTC TGC GTG AAC CTG GAG GGT GGC TAC AAG TGC CAG TGT GAG
pro asp thr cys ser gln leu cys val asn leu glu gly gly tyr lys cys gln cys glu
340/361 350/371
Exon 9
1141 1171 1187
GAA GGC TTC CAG CTG GAC CCC CAC ACG AAG GCC TGC AAG GCT GTG GGC TCC ATC GCC TAC
glu gly phe gln leu asp pro his thr lys ala cys lys ala val gly ser ile ala tyr
360/381 370/391
1201 1231
CTC TTC TTC ACC AAC CGG CAC GAG GTC AGG AAG ATG ACG CTG GAC CGG AGC GAG TAC ACC
leu phe phe thr asn arg his glu val arg lys met thr leu asp arg ser glu tyr thr
380/401 390/411
1261 1291
AGC CTC ATC CCC AAC CTG AGG AAC GTG GTC GCT CTG GAC ACG GAG GTG GCC AGC AAT AGA
ser leu ile pro asn leu arg asn val val ala leu asp thr glu val ala ser asn arg
400/421 410/431
Exon 10
1321 1351 1359
ATC TAC TGG TCT GAC CTG TCC CAG AGA ATG ATC TGC AGC ACC CAG CTT GAC AGA GCC CAC
ile tyr trp ser asp leu ser gln arg met ile cys ser thr gln leu asp arg ala his
420/441 430/451
1381 1411
GGC GTC TCT TCC TAT GAC ACC GTC ATC AGC AGG GAC ATC CAG GCC CCC GAC GGG CTG GCT
gly val ser ser tyr asp thr val ile ser arg asp ile gln ala pro asp gly leu ala
440/461 450/471
1441 1471
GTG GAC TGG ATC CAC AGC AAC ATC TAC TGG ACC GAC TCT GTC CTG GGC ACT GTC TCT GTT
val asp trp ile his ser asn ile tyr trp thr asp ser val leu gly thr val ser val
460/481 470/491
1501 1531
GCG GAT ACC AAG GGC GTG AAG AGG AAA ACG TTA TTC AGG GAG AAC GGC TCC AAG CCA AGG
ala asp thr lys gly val lys arg lys thr leu phe arg glu asn gly ser lys pro arg
480/501 490/511
Exon 11
1561 1587 1591
GCC ATC GTG GTG GAT CCT GTT CAT GGC TTC ATG TAC TGG ACT GAC TGG GGA ACT CCC GCC
ala ile val val asp pro val his gly phe met tyr trp thr asp trp gly thr pro ala
500/521 510/531
1621 1651
AAG ATC AAG AAA GGG GGC CTG AAT GGT GTG GAC ATC TAC TCG CTG GTG ACT GAA AAC ATT
lys ile lys lys gly gly leu asn gly val asp ile tyr ser leu val thr glu asn ile
520/541 530/551
Exon 12
1581 1706 1711
CAG TGG CCC AAT GGC ATC ACC CTA GAT CTC CTC AGT GGC CGC CTC TAC TGG GTT GAC TCC
gln trp pro asn gly ile thr leu asp leu leu ser gly arg leu tyr trp val asp ser
540/561 550/571
1741 1771
AAA CTT CAC TCC ATC TCA AGC ATC GAT GTC AAT GGG GGC AAC CGG AAG ACC ATC TTG GAG
lys leu his ser ile ser ser ile asp val asn gly gly asn arg lys thr ile leu glu
560/581 570/591
Exon 13
1801 1831 1846
GAT GAA AAG AGG CTG GCC CAC CCC TTC TCC TTG GCC GTC TTT GAG GAC AAA GTA TTT TGG
asp glu lys arg leu ala his pro phe ser leu ala val phe glu asp lys val phe trp
580/601 590/611
1861 1891
ACA GAT ATC ATC AAC GAA GCC ATT TTC AGT GCC AAC CGC CTC ACA GGT TCC GAT GTC AAC
thr asp ile ile asn glu ala ile phe ser ala asn arg leu thr gly ser asp val asn
600/621 610/631
1921 1951
TTG TTG GCT GAA AAC CTA CTG TCC CCA GAG GAT ATG GTC CTC TTC CAC AAC CTC ACC CAG
leu leu ala glu asn leu leu ser pro glu asp met val leu phe his asn leu thr gln
620/641 630/651
Exon 14
1981 1988 2011
CCA AGA GGA GTG AAC TGG TGT GAG AGG ACC ACC CTG AGC AAT GGC GGC TGC CAG TAT CTG
pro arg gly val asn trp cys glu arg thr thr leu ser asn gly gly cys gln tyr leu
640/661 650/671
2041 2071
TGC CTC CCT GCC CCG CAG ATC AAC CCC CAC TCG CCC AAG TTT ACC TGC GCC TGC CCG GAC
cys leu pro ala pro gln ile asn pro his ser pro lys phe thr cys ala cys pro asp
660/681 670/691
2041 2071
TGC CTC CCT GCC CCG CAG ATC AAC CCC CAC TCG CCC AAG TTT ACC TGC GCC TGC CCG GAC
cys leu pro ala pro gln ile asn pro his ser pro lys phe thr cys ala cys pro asp
660/681 670/691
Exon 15
2101 2131 2141
GGC ATG CTG CTG GCC AGG GAC ATG AGG AGC TGC CTC ACA GAG GCT GAG GCT GCA GTG GCC
gly met leu leu ala arg asp met arg ser cys leu thr glu ala glu ala ala val ala
680/701 690/711
2161 2191
ACC CAG GAG ACA TCC ACC GTC AGG CTA AAG GTC AGC TCC ACA GCC GTA AGG ACA CAG CAC
thr gln glu thr ser thr val arg leu lys val ser ser thr ala val arg thr gln his
700/721 710/731
2221 2251
ACA ACC ACC CGG CCT GTT CCC GAC ACC TCC CGG CTG CCT GGG GCC ACC CCT GGG CTC ACC
thr thr thr arg pro val pro asp thr ser arg leu pro gly ala thr pro gly leu thr
720/741 730/751
Exon 16
2281 2312
ACG GTG GAG ATA GTG ACA ATG TCT CAC CAA GCT CTG GGC GAC GTT GCT GGC AGA GGA AAT
thr val glu ile val thr met ser his gln ala leu gly asp val ala gly arg gly asn
740/761 750/771
Exon 17
2341 2371 2390
GAG AAG AAG CCC AGT AGC GTG AGG GCT CTG TCC ATT GTC CTC CCC ATC GTG CTC CTC GTC
glu lys lys pro ser ser val arg ala leu ser ile val leu pro ile val leu leu val
760/781 770/791
2401 2431
TTC CTT TGC CTG GGG GTC TTC CTT CTA TGG AAG AAC TGG CGG CTT AAG AAC ATC AAC AGC
phe leu cys leu gly val phe leu leu trp lys asn trp arg leu lys asn ile asn ser
780/801 790/811
2461 2491
ATC AAC TTT GAC AAC CCC GTC TAT CAG AAG ACC ACA GAG GAT GAG GTC CAC ATT TGC CAC
ile asn phe asp asn pro val tyr gln lys thr thr glu asp glu val his ile cys his
800/821 810/831
Exon 18
2521 2548 2580
AAC CAG GAC GGC TAC AGC TAC CCC TCG AGA CAG ATG GTC AGT CTG GAG GAT GAC GTG GCG
asn gln asp gly tyr ser tyr pro ser arg gln met val ser leu glu asp asp val ala
820/841
The translated sequence of LDLR, the exons, the Arabic mutation at position
1706-2 A > T in the intervening sequence 11 (not shown). SEQ ID NO 3
corresponds to the cDNA while SEQ ID NO 4 is the translated protein sequence
http://www.umd.necker.fr/LDLR/gene.sequence.html
5′-CAG CTA TTC TCT GTC CTC CCA CCA G (SEQ ID NO: 5)
5′-CGTACGAGATGCAAGCACTTAGGTG (SEQ ID NO: 6);
5′-CCAGGTGCTTTTCTGCTAGG (SEQ ID NO: 7)
5′-TCACTCCATCTCAAGCATCG (SEQ ID NO: 8)
5′-CCTCTCCAGGTGCTTTTCTG (SEQ ID NO: 9)
```

1. A method of identifying individuals susceptible to familial hypercholesterolemia comprising:
identifying in a sample from said individual at least one polymorphism at position 1706-2 of the coding region (41902 of the genomic DNA) in the low density lipoprotein receptor gene, and wherein the presence of at least one said polymorphism is indicative of said individual being of a higher susceptibility to familial hypercholesterolemia.

2. The method according to claim 1 and wherein said polymorphism corresponds to a substitution at said position 1706-2.

3. The method according to claim 1, wherein said polymorphism corresponds to a substitution of the nucleotide from A to T.

4. The method according to claim 1 wherein said identification of said polymorphism is carried out by any of polymerase chain reaction, hybridization, Southern blotting onto membrane, digestion with nucleases, restriction fragment length polymorphism, or direct sequencing, flowcytometry, Western Blotting or other immunological techniques or combinations thereof.

5. The method according to claim 1 wherein said identification step comprises using primer combinations including at least one of SEQ ID NOs: 5 and 6, 7, 8 and 9

6. An isolated nucleic acid molecule comprising a nucleotide sequence encoding a mutated LDL receptor comprising an amino acid sequence exhibiting at least 70%, 75%, 80%, 85% 90%, 95% or 99% sequence identity or homology to the amino acid sequence according to SEQ ID NO. 4.

7. An isolated nucleic acid molecule according to claim 6 wherein said nucleotide sequence comprises a mutated LDLR gene having a single nucleotide polymorphism at position 1706-2 of the coding region (41902 of the Genomic sequence) according to SEQ ID No 2.

8. An isolated nucleic acid molecule according to claim 6, wherein said polymorphism comprises a substitution at said position 1706-2.

9. An isolated nucleic acid molecule according to any of claim 6, wherein said polymorphism comprises a substitution in the wild type LDLR gene at position 1706-2 of the coding region and which is a substitution of nucleotide A to T.

10. An isolated nucleic acid molecule according to claim 6, wherein said nucleic acid is DNA or RNA.

11. An isolated nucleic acid molecule according to claim 10, wherein said DNA is a cDNA molecule.

12. An isolated nucleic acid molecule according to claim 6 wherein said nucleic acid molecule is a mammalian nucleic acid molecule.

13. An isolated nucleic acid molecule comprising a nucleotide sequence exhibiting at least 70%, 75%, 80%, 85%, 90%, 95% or 99% sequence identity or homology to the nucleic acid sequence according to SEQ ID NO. 3.

14. An isolated amino acid molecule comprising an amino acid sequence exhibiting at least 70%, 75%, 80%, 85%, 90%, 95% or 99% sequence identity or homology to the amino acid sequence according to SEQ ID NO. 4.

15. A recombinant expression vector suitable for transformation of a host cell comprising a nucleic acid as in claim 6.

16. The recombinant expression vector of claim 15, wherein the recombinant expression vector is a plasmid.

17. The recombinant expression vector of claim 15, wherein the recombinant expression vector is a prokaryotic or eukaryotic expression vector.

18. The recombinant expression vector of claim 15, wherein the nucleic acid molecule is operatively linked to a regulatory or expression control sequence.

19. A transformed host cell comprising the recombinant expression vector of claim 15.

20. A transformed host cell according to claim 19, wherein the host cell is a eukaryotic or prokaryotic host cell.

21. An isolated polypeptide encoded by a nucleic acid of claim 6.

22. An antibody or antigen-binding fragment thereof specific for an epitope of a protein as claimed in claim 14.

23. The antibody or antigen-binding fragment of claim 22 which is a monoclonal antibody or a polyclonal antibody.

24. A nucleic acid probe which hybridizes specifically to a nucleic acid according to claim 6 under conditions of stringency that prevents it from hybridizing to wild-type DNA.

25. A nucleic acid probe according to claim 24, wherein said probe comprises a nucleic acid sequence complementary to the sequence of said LDLR gene incorporating said nucleotide substitution at position 1706-2 (41902 of the genomic DNA) of SEQ ID NO:2.

26. A recombinant expression vector suitable for transformation of a host cell comprising a nucleic acid as in claim 13.

27. The recombinant expression vector of claim 26, wherein the recombinant expression vector is a plasmid.

28. The recombinant expression vector of claim 26, wherein the recombinant expression vector is a prokaryotic or eukaryotic expression vector.

29. The recombinant expression vector of claim 26, wherein the nucleic acid molecule is operatively linked to a regulatory or expression control sequence.

30. A transformed host cell comprising the recombinant expression vector of claim 26.

31. A transformed host cell according to claim 30, wherein the host cell is a eukaryotic or prokaryotic host cell.

32. An isolated polypeptide encoded by a nucleic acid of claim 13.

33. A nucleic acid probe which hybridizes specifically to a nucleic acid according to claim 13 under conditions of stringency that prevents it from hybridizing to wild-type DNA.

34. A nucleic acid probe according to claim 33, wherein said probe comprises a nucleic acid sequence complementary to the sequence of said LDLR gene incorporating said nucleotide substitution at position 1706-2 (41902 of the genomic DNA) of SEQ ID NO:2.


SEQUENCE LISTING (67 sequences)

SEQ ID NO: 1 - DNA, 1140 bp, Homo sapiens
gcctgagcctggctgtttcttccagaattcgttgcacgcattggctgggatcctcccccgccctccagcctcacagctattctctgtcctcccaccagcttcatgtactggactgactggggaactcccgccaagatcaagaaagggggcctgaatggtgtggacatctactcgctggtgactgaaaacattcagtggcccaatggcatcaccctaggtatgttcgcaggacagccgtcccagccagggccgggcacaggctggaggacagacgggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtggccaggccctcaggaccctctgggactggcatcagcacgtgacctctccttatccacttgtgtgtctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttct

SEQ ID NO: 2 - DNA, 1140 bp, Homo sapiens
gcctgagcctggctgtttcttccagaattcgttgcacgcattggctgggatcctcccccgccctccagcctcacagctattctctgtcctcccaccagcttcatgtactggactgactggggaactcccgccaagatcaagaaagggggcctgaatggtgtggacatctactcgctggtgactgaaaacattcagtggcccaatggcatcaccctaggtatgttcgcaggacagccgtcccagccagggccgggcacaggctggaggacagacgggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtggccaggccctcaggaccctctgggactggcatcagcacgtgacctctccttatccacttgtgtgtcttgatctcctcagtggccgcctctactgggttgactccaaacttcactccatctcaagcatcgatgtcaacgggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttct

SEQ ID NO: 3 - DNA, 2580 bp, Homo sapiens
atggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcg

SEQ ID NO: 4 - protein, 860 aa, Homo sapiens [sequence omitted]

SEQ ID NO: 5 - DNA, 25 bp, Homo sapiens
cagctattctctgtcctcccaccag

SEQ ID NO: 6 - DNA, 25 bp, Homo sapiens
cgtacgagatgcaagcacttaggtg

SEQ ID NO: 7 - DNA, 20 bp, Homo sapiens
ccaggtgcttttctgctagg

SEQ ID NO: 8 - DNA, 20 bp, Homo sapiens
tcactccatctcaagcatcg

SEQ ID NO: 9 - DNA, 20 bp, Homo sapiens
cctctccaggtgcttttctg

SEQ ID NO: 10 - DNA, 25 bp, Homo sapiens
gagtgggaatcagagcttcacgggt

SEQ ID NO: 11 - DNA, 25 bp, Homo sapiens
ccacgtcatttacagcatttcaatg

SEQ ID NO: 12 - DNA, 25 bp, Homo sapiens
actcctccccctgctagaaacctca

SEQ ID NO: 13 - DNA, 25 bp, Homo sapiens
ttctggcgcctggagcaagccttac

SEQ ID NO: 14 - DNA, 25 bp, Homo sapiens
cctttctccttttcctctctctcag

SEQ ID NO: 15 - DNA, 25 bp, Homo sapiens
aaaataaatgcatatcatgcccaaa

SEQ ID NO: 16 - DNA, 25 bp, Homo sapiens
tgacagttcaatcctgtctcttctg

SEQ ID NO: 17 - DNA, 25 bp, Homo sapiens
atagcaaaggcagggccacacttac

SEQ ID NO: 18 - DNA, 25 bp, Homo sapiens
gttgggagacttcacacggtgatgg

SEQ ID NO: 19 - DNA, 25 bp, Homo sapiens
acttaggcagtggaactcgaaggcc

SEQ ID NO: 20 - DNA, 25 bp, Homo sapiens
ccccagctgtgggcctgcgacaacg

SEQ ID NO: 21 - DNA, 25 bp, Homo sapiens
gggggagcccagggacaggtgatag

SEQ ID NO: 22 - DNA, 25 bp, Homo sapiens
caacacactctgtcctgttttccag

SEQ ID NO: 23 - DNA, 25 bp, Homo sapiens
ggaaaaccagatggccagcgctcac

SEQ ID NO: 24 - DNA, 25 bp, Homo sapiens
tccttcctctctctggctctcacag

SEQ ID NO: 25 - DNA, 25 bp, Homo sapiens
gcaagccgcctgcaccgagactcac

SEQ ID NO: 26 - DNA, 25 bp, Homo sapiens
agtctgcatccctggccctgcgcag

SEQ ID NO: 27 - DNA, 25 bp, Homo sapiens
agggctcagtccaccggggaatcac

SEQ ID NO: 28 - DNA, 25 bp, Homo sapiens
ccaagcctctttctctctcttccag

SEQ ID NO: 29 - DNA, 25 bp, Homo sapiens
ccacccgccgccttcccgtgctcac

SEQ ID NO: 30 - DNA, 25 bp, Homo sapiens
tccatcgacgggtcccctctgaccc

SEQ ID NO: 31 - DNA, 25 bp, Homo sapiens
agccctcatctcacctgcgggccaa

SEQ ID NO: 32 - DNA, 25 bp, Homo sapiens
agatgagggctcctggtgcgatgcc

SEQ ID NO: 33 - DNA, 25 bp, Homo sapiens
gcccttggtatccgcaacagagaca

SEQ ID NO: 34 - DNA, 25 bp, Homo sapiens
gatccacagcaacatctactggacc

SEQ ID NO: 35 - DNA, 25 bp, Homo sapiens
agccctcagcgtcgtggatacgcac

SEQ ID NO: 36 - DNA, 25 bp, Homo sapiens
cagctattctctgtcctcccaccag

SEQ ID NO: 37 - DNA, 25 bp, Homo sapiens
tgggacggctgtcctgcgaacatac

SEQ ID NO: 38 - DNA, 25 bp, Homo sapiens
gcacgtgacctctccttatccactt

SEQ ID NO: 39 - DNA, 25 bp, Homo sapiens
cacctaagtgcttcgatctcgtacg

SEQ ID NO: 40 - DNA, 25 bp, Homo sapiens
gtcatcttccttgctgcctgtttag

SEQ ID NO: 41 - DNA, 25 bp, Homo sapiens
gtttccacaaggaggtttcaaggtt

SEQ ID NO: 42 - DNA, 25 bp, Homo sapiens
cctgactccgcttcttctgccccag

SEQ ID NO: 43 - DNA, 25 bp, Homo sapiens
cgcagaaacaaggcgtgtgccacac

SEQ ID NO: 44 - DNA, 25 bp, Homo sapiens
gaagggcctgcaggcacgtggcact

SEQ ID NO: 45 - DNA, 25 bp, Homo sapiens
gtgtggtggcgggcccagtctttac

SEQ ID NO: 46 - DNA, 25 bp, Homo sapiens
cctcactcttgcttctctcctgcag

SEQ ID NO: 47 - DNA, 25 bp, Homo sapiens
cgctgggggaccggcccgcgcttac

SEQ ID NO: 48 - DNA, 25 bp, Homo sapiens
tgacagagcgtgcctctccctacag

SEQ ID NO: 49 - DNA, 25 bp, Homo sapiens
gctttctagagagggtcacactcac

SEQ ID NO: 50 - DNA, 25 bp, Homo sapiens
tccgctgtttaccatttgttggcag

SEQ ID NO: 51 - DNA, 25 bp, Homo sapiens
aataaaacaaggccggcgaggtctc

SEQ ID NO: 52 - DNA, 20 bp, Homo sapiens
ctaggacacagcaggtcgtg

SEQ ID NO: 53 - DNA, 19 bp, Homo sapiens
ggagctgttgcactggaag

SEQ ID NO: 54 - DNA, 20 bp, Homo sapiens
acgatgggaagtgcatctct

SEQ ID NO: 55 - DNA, 20 bp, Homo sapiens
ttgatgggttcatctgacca

SEQ ID NO: 56 - DNA, 20 bp, Homo sapiens
accgggaatatgactgcaag

SEQ ID NO: 57 - DNA, 18 bp, Homo sapiens
tcctcaggttggggatga

SEQ ID NO: 58 - DNA, 19 bp, Homo sapiens
tggagggtggctacaagtg

SEQ ID NO: 59 - DNA, 20 bp, Homo sapiens
gagttccccagtcagtccag

SEQ ID NO: 60 - DNA, 21 bp, Homo sapiens
gtctctgttgcggataccaag

SEQ ID NO: 61 - DNA, 21 bp, Homo sapiens
cctctcacaccagttcactcc

SEQ ID NO: 62 - DNA, 22 bp, Homo sapiens
gccgtctttgaggacaaagtat

SEQ ID NO: 63 - DNA, 20 bp, Homo sapiens
cacgctactgggcttcttct

SEQ ID NO: 64 - DNA, 20 bp, Homo sapiens
aggacacagcacacaaccac

SEQ ID NO: 65 - DNA, 21 bp, Homo sapiens
tctgcctcccagatgaataaa

SEQ ID NO: 66 - DNA, 20 bp, Homo sapiens
tcagtctggaggatgacgtg

SEQ ID NO: 67 - DNA, 20 bp, Homo sapiens
caaaggctaacctggctgtc